SEQUENCE LISTING (11 sequences)

SEQ ID NO: 1 - RNA, 21 nt, Artificial Sequence
  synthesized siRNA sequences - oligo-1 (sense sequence)
gcuuaccgucagauacaaguu

SEQ ID NO: 2 - RNA, 21 nt, Artificial Sequence
  synthesized siRNA sequences - oligo-1 (antisense sequence)
cuuguaucugacgguaagcuu

SEQ ID NO: 3 - RNA, 21 nt, Artificial Sequence
  synthesized siRNA sequences - oligo-2 (sense sequence)
gacggcuaaguccuuguuuuu

SEQ ID NO: 4 - RNA, 21 nt, Artificial Sequence
  synthesized siRNA sequences - oligo-2 (antisense sequence)
aaacaaggacuuagccgucuu

SEQ ID NO: 5 - RNA, 21 nt, Artificial Sequence
  synthesized siRNA sequences - oligo-3 (sense sequence)
acacuugagggccuguauguu

SEQ ID NO: 6 - RNA, 21 nt, Artificial Sequence
  synthesized siRNA sequences - oligo-3 (antisense sequence)
cauacaggcccucaaguguuu

SEQ ID NO: 7 - RNA, 21 nt, Artificial Sequence
  synthesized siRNA sequences - oligo-4 (sense sequence)
ccugcucucggaggaugccuu

SEQ ID NO: 8 - RNA, 21 nt, Artificial Sequence
  synthesized siRNA sequences - oligo-4 (antisense sequence)
ggcauccuccgagagcagguu

SEQ ID NO: 9 - DNA, 19 bp, Artificial Sequence
  synthesized forward primer for real-time RT-PCR
atgcctttggccattgttg

SEQ ID NO: 10 - DNA, 23 bp, Artificial Sequence
  synthesized reverse primer for real-time RT-PCR
acaatccacttctacagctgcct

SEQ ID NO: 11 - DNA, 14 bp, Artificial Sequence
  synthesized A3 MGB probe
tcagcctgggcatc

(19) United States
(12) Patent Application Publication
(10) Pub. No.: US 2006/0194756 A1
(43) Pub. Date: Aug. 31, 2006

(54) Enhancing treatment of HIF-1 mediated disorders with adenosine A3 receptor agonists

(76) Inventors: Pier Andrea Borea, Ferrara (IT); Pier Giovanni Baraldi, Ferrara (IT); Stefania Merighi, Ferrara (IT); Stephen MacLennan, Cary, NC (US); Edward Leung, Cary, NC (US); Allan Moorman, Durham, NC (US)

Correspondence Address: Christopher A. Klein, 400 Crossing Blvd., Bridgewater, NJ, 08807, US

(21) Appl. No.: 11/286,129

(22) Filed: Nov. 22, 2005

Related U.S. Application Data
(60) Provisional application No. 60/630,555, filed on Nov. 22, 2004.

(51) Int. Cl. A61K 31/7076

(52) U.S. Cl. 514/45; 514/46

(57) ABSTRACT

The present invention relates to the use of adenosine receptor agonists, preferably A₃ receptor agonists, either alone or in combination with other agents for the treatment, prevention and/or management of diseases or disorders associated with under-expression of HIF-1α and/or decreased HIF-1 α activity (e.g., ischemic related disorders). The methods of the invention are directed to methods of reducing tissue damage (e.g., substantially prevention tissue damage, inducing tissue protection) resulting from ischemia orhypoxia. The invention provides methods for treating, preventing and/or ameliorating one or more symptoms of hypoxic or HIF-1α related disorders by administering an A₃ receptor agonist either alone or in combination with other agents.

### 1. CROSS-REFERENCE TO RELATED APPLICATIONS

This application claims the benefit of U.S. Provisional Application No. 60/630,555, filed Nov. 22, 2004, the disclosure of which is hereby incorporated by reference in its entirety.

### 2. FIELD OF THE INVENTION

The present invention relates to the use of adenosine receptor agonists, preferably A₃ receptor agonists, either alone or in combination with other agents for the treatment, prevention and/or management of diseases or disorders associated with under-expression of HIF-1α and/or decreased HIF-1α activity (e.g., ischemic related disorders). The methods of the invention are directed to methods of reducing tissue damage (e.g., substantially preventing tissue damage, inducing tissue protection) resulting from ischemia or hypoxia. The invention provides methods for treating, preventing and/or ameliorating one or more symptoms of hypoxic or HIF-1α related disorders by administering an A₃ receptor agonist either alone or in combination with other agents.

### 3. BACKGROUND OF THE INVENTION

3.1 Adenosine

Adenosine, recently called a “primordial signalling molecule” (Linden 2001, Annu. Rev. Pharmacol. Toxicol, 41: 775-87), has the potential of influencing development, is present in and modulates physiological responses in all mammalian tissues. The actions of adenosine are most prominent in tissues where oxygen demand is high and there is reduction in oxygen tension, i.e., within solid tumors, where cell proliferation is greater than the rate of blood vessel formation (Sitkovsky, 2004 Annu. Rev. Immunol. 22, 657-82; Fredholm, 2001, Pharmacol. Rev. 53, 527-552). As a result, the tumor has local areas of hypoxia and adenosine accumulates to high levels (Hockel, 2001, J. Natl. Cancer Inst. 93, 266-76). In particular, it is recognized that significant levels of adenosine are present in the extracellular fluid of solid tumors (Blay, 1997, Cancer Res., 57, 2602-5), suggesting a role for this nucleoside in tumor growth.

Adenosine has been linked to tumor development. Increased adenosine concentration has been reported inside tumoral masses. It has been speculated that it represents the anti-tumor agent that prevents tumor growth in muscle tissue in vivo and that impairs malignant cell growth and survival in vitro. However, it is known that adenosine acts as cyto-protective agent during ischemic damage in brain and heart. Adenosine is known to be released in hypoxia. Numerous studies have shown adenosine to protect cells in the heart from ischemic damage.

Adenosine has been shown to have protective roles in numerous animal models and in man (Am. J. Cardiol. 79(12A):44-48 (1997). For example, in the heart, both the A₁ and A₃ receptors offer protection against ischemia (Am. J. Physiol., 273(42)H501-505 (1997)). However, it is the A₃ receptor that offers sustained protection against ischemia (PNAS 95:6995-6999 (1998)). The ability of adenosine to protect tumor cells against hypoxia has not been recognized by others prior to the instant invention.

Adenosine interacts with cell surface receptors that are glycoproteins coupled to different members of G protein family. By now four adenosine receptors have been cloned and characterised: A₁, A_{2A}, A_{2B} and A₃. Selective antagonists for the A₃ receptor have been proposed for use as anti-inflammatory and antiischemic agents in the brain. Recently, A₃ antagonists have been under development as antiasthmatic, antidepressant, anti-arrhythmic, renal protective, antiparkinson and cognitive enhancing drugs. For example, U.S. Pat. No. 5,646,156 to Marlene Jacobson et al. inhibits eosinophil activation by using selected A₃ antagonists.

Recent studies in myocytes have shown the adenosine A₃ receptors to be responsible for long-term protection against ischemia (Liang and Jacobson, PNAS, 1998, 95:6995-6999). While the present inventors have hypothesized that adenosine plays a protective role in other cell types, including tumor cells, in addition to myocytes, no efforts have been made to limit the protective effect of adenosine on tumor cells.

3.2 HIF-1 Biology

Hypoxia-inducible factor (HIF)-1 is a transcription factor that functions as a master regulator of oxygen homeostasis (Semenza, 2001, Trends Mol. Med. 7, 345-350.

HIF-1 is a heterodimer composed of an inducibly-expressed HIF-1α subunit and a constitutively-expressed HIF-1β subunit (Epstein, 2001, Cell, 107, 43-54). HIF-1α and HIF-1β mRNAs are constantly expressed under normoxic and hypoxic conditions (Wiener, 1996 Biochem. Biophys. Res. Commun. 225,485-488). The unique feature of HIF-1 is the regulation of HIF-1α expression: it increases as the cellular O₂ concentration is decreased (Cramer, 2003, Cell, 112, 645-657, Pugh, 2003, Nat. Med. 9, 677-84). During normoxia, HIF-1α is rapidly degraded by the ubiquitin proteasome system, whereas exposure to hypoxic conditions prevents its degradation (Minchenko, 2002 J. Biol. Chem., 277, 6183-6187; Semenza, 2000, J. Appl. Physiol., 88, 1474-1480).

A growing body of evidence indicates that HIF-1 contributes to tumor progression and metastasis (Hopfl, 2004, Am. J. Physiol. Regul. Integr. Comp. Physiol. 286, R608-23; Welsh, 2003, Curr. Cancer Drug Targets. 3, 391-405). Immunohistochemical analyses have shown that HIF-1α is present in higher levels in human tumors than in normal tissues (Zhong, 2000, Cancer Res. 60, 1541-5). Tumor progression is associated with adaptation to hypoxia, and there is an inverse correlation between tumor oxygenation and clinical outcome (Pugh, 2003, Ann. Med. 35, 380-90; Semenza, 2000 J. Appl. Physiol., 88, 1474-1480). In particular, the levels of HIF-1 activity in cells are correlated with tumorigenicity and angiogenesis in nude mice (Chen, 2003, Am. J. Pathol. 162,1283-91). Tumor cells lacking HIF-1 expression are markedly impaired in their growth and vascularization (Carmeliet, 1998, Nature 394, 485-90; Jiang, 1997, Cancer Res., 57, 5328-5335; Maxwell, 1997, Proc. Natl. Acad. Sci. U.S.A., 94, 8104-8109; Ryan, 1998 EMBO J. 17, 3005-3015). Therefore, since HIF-1α expression and activity appear central to tumor growth and progression, HIF-1 inhibition has become an appropriate anticancer target (Kung, 2000, Nat. Med. 6, 1335-40).

HIF-1α has also been implicated in other diseases including ischemic cardiovascular diseases, pulmonary hypertension, and pregnancy disorders.

### 4. SUMMARY OF THE INVENTION

Although not intending to be bound by a particular mechanism of action, the invention is based, in part, on the inventor's discovery that adenosine regulates HIF-1α levels via A₃ receptor, therefore activating this pathway with A₃ receptor agonists would have beneficial effects in diseases where HIF-1α expression and/or activity is impaired. Accordingly, the present invention relates to methods for the treatment, prevention, and/or management of diseases or disorders associated with decreased expression of HIF-1α and/or decreased HIF-1α activity (e.g., ischemic cardiac disorders) by using A₃ receptor agonists. The methods of the invention may be employed in combination with A₁, A_{2A}, or A_{2B} receptor agonists. Although not intending to be bound by a particular mechanism of action the A₃ receptor agonists of the invention up-regulate HIF-1α expression and thus promote angiogenesis and reversal of ischemic damage as a result of low level of HIF-1α expression and/or activity. In most preferred embodiments, the methods of the invention relate to treatment, prevention, and/or management of diseases or disorders associated with a reduced expression of HIF-1α and/or decreased HIF-1α activity by using A₃ receptor agonists alone.

The invention provides methods for treatment of HIF-1-mediated disorders, including hypoxia- or ischemia-related tissue damage, which are improved or ameliorated by modulation of HIF-1 expression or activity. The relevant clinical conditions treated by the methods and compositions of the invention include ischemia due to a disease of the cerebral, coronary, or peripheral circulation. One therapeutic goal of the invention is to promote angiogenesis in the ischemic tissue by enhancing HIF-1α expression and/or activity. Although not intending to be bound by a particular mechanism of action, such an enhancement may result in dimerization of HIF-1α with endogenous HIF-1β, binding to specific DNA sequences, and activation transcription of hypoxia-inducible genes relevant to angiogenesis, such as, but not limited to, the gene encoding vascular endothelial growth factor (VEGF), a known HIF-1 target gene.

In other embodiments, the methods of the invention provide prophylactic measures to induce angiogenesis in patients at risk of ischemic disease, even if there is no hypoxia at the time, in order to prevent ischemic conditions, e.g., heart attacks.

The methods of the invention are directed to methods of reducing tissue damage (e.g., substantially preventing tissue damage, inducing tissue protection) resulting from ischemia or hypoxia comprising administering to a mammal in need of such treatment a therapeutically effective amount of an A₃ receptor agonist, a pharmaceutically acceptable salt of said compound. Ischemic or hypoxic tissues thay may benefit from the the methods and compositions of the invention include without limitation cardiac, brain, liver, kidney, lung, gut, skeletal muscle, spleen, pancreas, nerve, spinal cord, retina tissue, the vasculature, or intestinal tissue. An especially preferred ischemic or hypoxic tissue is cardiac tissue. It is especially preferred that the compounds are administered to prevent perioperative myocardial ischemic injury. In some embodiments, the compounds of this invention are administered prophylactically. The invention encompasses management of ischemic or hypoxic tissue damage that occurs during organ transplantation. Preferably, the compounds of this invention are administered prior to, during or shortly after, cardiac surgery or non-cardiac surgery.

Another aspect of this invention is directed to methods of reducing myocardial tissue damage (e.g., substantially preventing tissue damage, inducing tissue protection) during surgery (e.g., coronary artery bypass grafting (CABG) surgeries, vascular surgeries, percutaneous transluminal coronary angioplasty (PTCA) or any percutaneous transluminal coronary intervention (PTCI), organ transplantation, or other non-cardiac surgeries) comprising administering to a mammal a therapeutically effective amount of a compound of an A₃ receptor agonist.

The invention encompasses treating and/or preventing ischemic heart disease using one or more compounds of the invention either alone or in combination with other therapeutic and/or prophylactic agents. Although not intending to be bound by a particular mechanism of action ischemia is often caused by a reduction in coronary blood flow relative to myocardial demand. The reduction in blood flow may result from a variety of reasons, and typically occurs as a result of atherosclerosis. The methods of the invention are effective in reducing ischemic related impaired blood flow or other ischemic related tissue or organ damage including damage to the heart muscle, cardiac arrhythmias, angina, myocardial infarction, congestive heart failure, and sudden cardiac death. Ischemia may be assessed by any method known to those skilled in the art and disclosed herein. An assessment of ischemic damage may be made, for example, by measuring the infarct (scar) size of the organ.

The compounds and methods of the invention are particularly useful for increasing vasogenesis or angiogenesis to treat diseases or conditions associated with insufficient vascularization, or an injury to vessels. For example, the A₃ receptor agonist compounds of the invention may be administered to individuals having undergone surgery, particularly vessel or cardiac surgery, to improve the rate of vessel repair. In a second example, the agonist compounds of the invention may be used to treat individuals having insufficient peripheral blood flow, such as individual having a non-healing wound, or Reynaud's disease. Thus, in another embodiment, the invention provides a method of treating an individual, wherein said individual has a condition or disease associated with insufficient angiogenesis or vasogenesis, comprising administering to said individual an amount of an agent that detectably increases angiogenesis or vasogenesis, said agent administered in an amount sufficient to increase said angiogenesis or vasogenesis.

The methods and compositions of the invention comprising A₃ receptor agonists are particularly useful when the levels of HIF-1α expression and/or activity are reduced below the standard or background level, as determined using methods known to those skilled in the art and dislosed herein.

As used herein, “restoration” of a measured level of HIF-1α to a standard level means that the amount or concentration of HIF-1α in a sample or subject is lower in a subject or sample relative to the standard as detected by any method now known in the art or to be developed in the future for measuring HIF-1α levels and the methods of the invention allow the level to return to the background or standard level. Such a restoration may include, but is not limited to a restoration of HIF-1α level to a level which is about a 10%, about a 20%, about a 40%, about an 80%, about a 2-fold, about a 4-fold, about a 10-fold, about a 20-fold, about a 50-fold, about a 100-fold, about a 2 to 20 fold, 2 to 50 fold, 2 to 100 fold, 20 to 50 fold, 20 to 100 fold within the standard level. The term “about” as used herein, refers to levels of elevation of the standard numerical value plus or minus 10% of the numerical value.

The therapeutic methods of the invention comprise administering a therapeutically effective amount of an A₃ receptor agonist to improve the therapeutic efficacy of diseases or disorders associated with reduced expression of HIF-1α and/or decreased HIF-1α activity (e.g., ischemic cardiac disorders) relative to the traditional modes of such therapies.

Preferably the methods of the invention increase the HIF-1α level to the background level within at least 1 day, 1 week, one month, 2 months, at least 4 months, at least 6 months of the therapeutic regime. In a most preferred embodiment, the methods of the invention result in a complete restoration of HIF-1α level to the standard level. The invention encompasses restoration of the HIF-1α level to a level which is within about about 10%, about 20%, about 30%, about 40%, about 50% of the background level.

In a preferred specific embodiment, the invention encompasses a method for treatment, prevention and/or management of diseases or disorders associated with under-expression of HIF-1α and/or decreased HIF-1α activity (e.g., ischemic disorders) comprising administering a therapeutically and/or prophylactically effective amount of an A₃ receptor agonist compound as disclosed herein.

The A₃ receptor agonists of the invention, either alone or in combination with other therapeutic and prophylactic agents (including A₁ receptor agonists) are particularly useful for reducing hypoxia or ischemia-related tissue damage in a subject having or at risk of such damage. The invention relates to methods and compositions for the treatment, prevention and/or management of a HIF-1α mediated disease or disorder by using an A₃ receptor agonist of the invention. The methods of the invention contemplate administering a therapeutically and/or prophylactically effective amount of the A₃ receptor agonists alone or in combination with other therapeutic and/or prophylactic agents, including but not limited to A₁, A_{2A}, and A_{2B} agonists. The A₃ receptor agonists are particularly useful for the treatment of HIF-1α associated disease or disorder including without limitation, ischemic cardiovascular disorders (e.g., myocardial ischemia, cerebral ischemia, retinal ischemia), pulmonary hypertension, pregnancy disorders, (e.g., preeclampsia, intrauterine growth retardation), any surgical procedures wherein the blood supply needs to be shut off, or any other disorder with impaired blood flow. Although not intending to be bound by a particular mechanism of action the agonists of the invention are therapeutically effective by increasing the level and/or activity of HIF-1α or HIF-1α related activity which will promote angiogenesis. Overexpression of HIF-1α leads to dimerization with endogenous HIF-1β and activation of hypoxia inducible genes relevant to angiogenesis including but not limited to vascular endothelial growth factor.

The invention encompasses compounds which are A₃ receptor agonists for use in the methods of the invention. Examples of such compounds are disclosed in U.S. Patent Application Publication Nos. 20040204481 A1; 20040198693 A1; 20040198693 A1; 20040116376 A1; 20040106572 A1; 20030166605 A1; 20030143282 A1; 20030078232 A1; 20020165197; 20020115635 and U.S. Pat. Nos. 6,586,413; 6,448,253; 6,407,236; 6,358,964; 6,329,349; 6,211,165; 5,573,772; and 5,443,836; all of which are incorporated herein by reference in their entireties.

The present invention encompasses therapies which involve administering one or more of the compounds of the invention, to an animal, preferably a mammal, and most preferably a human, for preventing, treating, or ameliorating symptoms associated with a disease or disorder associated with hypoxia-inducible factor 1-α (HIF-1α).

The invention further provides a pharmaceutical composition comprising a therapeutically or prophylactically effective amount of a compound that specifically binds and activates an A₃ receptor and a pharmaceutically acceptable carrier The compounds can be used in a pharmaceutical formulation that also includes an adenosine A₁ agonist and one or more excipients.

The invention also encompasses a method for determining the prognosis of a a disease or disorder associated with under-expression of HIF-1α and/or decreased HIF-1α activity in a subject. Preferably, the subject is human, and most preferably the subject has been previously treated with a therapy regimen. The invention encompasses measuring at least a level of HIF-1α in a subject to determine if the subject is in need of the therapeutic and or prophylcatic methods of the inventon. The invention encompasses measuring a level of HIF-1 α in a sample obtained from the subject and comparing the level measured to a standard level, wherein restoration of the measured level of HIF-1 α relative to the standard level indicates that the subject is at an increased risk for progression of the disease or disorder, e.g., ischemic disorder.

4.1 Definitions

As used herein, the term “adenosine A₃ receptor agonist” is used to define a compound which is selective for the adenosine A₃ receptor, with an affinity for the adenosine A₃ receptor at least 10, and preferably, at least 50 times higher than the affinity for the adenosine A₁ and A₂ receptors. Specific and non-specific A₁, A₂ and A₃ receptor agonists are well known to those of skill in the art. Examples of these agonists are found, for example, in the 1999 RBI (Sigma) and Tocris catalogs. Examples of suitable agonists include without limitation AB-MECA (A₃), adenosine amine congener (ADAC) (A₁), N6-2-(4-aminophenyl)ethyl-adenosine (APNEA) (A₃), CGS-21680 HCl (A₂ₐ), 2-chloroadenosine (A₁>A₂), 2-chlorocyclopentyladenosine (A₁), N6-cyclohexyladenosine (A₁), N6-cyclopentyladenosine (A₁), 5′-N-cyclopropyl)-carboxamidoadenosine (A₂), DPMA (PD 125,944) (A₂ₐ), ENBA (S-) (A₁), 5′-N-ethylcarboxamidoadenosine (NECA) (A_{2b}), IB-MECA (A₃), MECA (A₂>A₁), 1-methylisoguanosine (A₁), metrifudil (A₂), 2-phenylaminoadenosine (A₂>A₁), N6-phenyladenosine (A₁>A₂), N6-phenylethyladenosine (A₁>A₂), R-PIA (A₁), S-PIA (A₁), N6-sulfophenyladenosine (A₁), and 2-chloro-IB-MECA (A₃).

Another example of an A₃ receptor agonist is a compound of the following general formula:

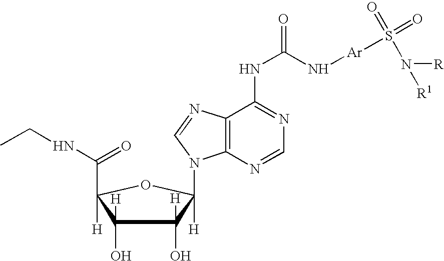

wherein Ar is an aryl group;

and R and R¹ are independently H, alkyl, aryl, substituted alky, substituted aryl, heteroaryl, alkenyl, substituted alkenyl, cycloalkenyl, substituted cycloalkenyl, cycloalkyl, substituted cycloalkyl, alkoxy, substituted alkoxy, alkynyl, substituted alkynyl, and where taken together R and R¹ may form a substituted or unsubstituted carbocyclic or heterocyclic fused ring system which includes both alicyclic and aromatic structures;

or a pharmaceutically acceptable salt thereof.

As used herein, a compound is selective for the A₃ receptor if its affinity at the A₃ receptor is greater than its affinity at the A₁, A₂ₐ and A_{2b} receptors. Preferably, the ratio of A₁/A₃ and A₂/A₃ affinity is greater than about 50, preferably between 50 and 100, and more preferably, greater than about 100. Since the pharmacology at the A₃ receptor varies between species, especially between rodent A₃ and human A₃ receptors, it is important to determine the selectivity of the A₃ compounds in human adenosine receptors. The same holds true for adenosine A₁ and A₂ₐ receptors in terms of whether they are selective.

As used herein, “restoration” of a measured level of HIF-1α to a standard level means that the amount or concentration of HIF-1α in a sample or subject is lower in a subject or sample relative to the standard as detected by any method now known in the art or to be developed in the future for measuring HIF-1α levels and the methods of the invention allow the level to return to the background or standard level. Such a restoration may include, but is not limited to a restoration of HIF-1α level to a level which is about a 10%, about a 20%, about a 40%, about an 80%, about a 2-fold, about a 4-fold, about an 10-fold, about a 20-fold, about a 50-fold, about a 100-fold, about a 2 to 20 fold, 2 to 50 fold, 2 to 100 fold, 20 to 50 fold, 20 to 100 fold within the standard level. The term “about” as used herein, refers to levels of elevation of the standard numerical value plus or minus 10% of the numerical value.

The term “standard level” or “background level” as used herein refers to a baseline amount of an HIF-1α level as determined in one or more normal subjects, i.e., a subject with no known history of past or current diseases, disorders. For example, a baseline may be obtained from at least one subject and preferably is obtained from an average of subjects (e.g., n=2 to 100 or more), wherein the subject or subjects have no prior history of a disease or disorder, especially no prior history of diseases associated with HIF-1α. In the present invention, the measurement of an HIF-1α level may be carried out using a HIF-1α probe or a HIF-1α activity assay (see Section 6.3.3).

As used herein “hypoxia” or “ischemia” referes to any condition whereby the physiology of the tissue is compromised and/or blood supply in one or more tissues is compromised. These two terms may be used interchangeably. The condition also encompasses any reduction in partial pressure of O₂ in one or more tissues. The term “hypoxia” or “ischemia” encompasses any condition in which a cell, tissue or organ experiences a lack of oxygen or reduced blood flow.

As used herein, the terms “treat,” “treating” and “treatment” refer to the eradication, removal, modification, or control of the disease that results from the administration of one or more therapeutic agents. In certain embodiments, such terms refer to the minimizing or delaying the progression of disease resulting from the administration of one or more therapeutic agents to a subject with such a disease.

As used herein, a “therapeutically effective amount” refers to that amount of the therapeutic agent sufficient to delay or minimize the spread of disease. A therapeutically effective amount may also refer to the amount of the therapeutic agent that provides a therapeutic benefit in the treatment or management of a disease or disorder, e.g., ischemic disease Further, a therapeutically effective amount with respect to a therapeutic agent of the invention means that amount of therapeutic agent alone, or in combination with other therapies, that provides a therapeutic benefit in the treatment or management of a disease. Used in connection with an amount of a compound of the invention, the term can encompass an amount that improves overall therapy, reduces or avoids unwanted effects, or enhances the therapeutic efficacy of or synergies with another therapeutic agent.

As used herein, the terms “prophylactic agent” and “prophylactic agents” refer to any agent(s) which can be used in the prevention of a disorder, or prevention of recurrence or spread of a disorder. A prophylactically effective amount may refer to the amount of prophylactic agent sufficient to prevent the recurrence or spread of disease, or the occurrence of such in a patient, including but not limited to those predisposed to a disease. A prophylactically effective amount may also refer to the amount of the prophylactic agent that provides a prophylactic benefit in the prevention of disease. Further, a prophylactically effective amount with respect to a prophylactic agent of the invention means that amount of prophylactic agent alone, or in combination with other agents, that provides a prophylactic benefit in the prevention of disease. Used in connection with an amount of a compound of the invention, the term can encompass an amount that improves overall prophylaxis or enhances the prophylactic efficacy of or synergies with another prophylactic agent, such as but not limited to a therapeutic antibody.

As used herein, the terms “manage,” “managing” and “management” refer to the beneficial effects that a subject derives from administration of a prophylactic or therapeutic agent, which does not result in a cure of the disease. In certain embodiments, a subject is administered one or more prophylactic or therapeutic agents to “manage” a disease so as to prevent the progression or worsening of the disease.

As used herein, the terms “prevent”, “preventing” and “prevention” refer to the prevention of the recurrence or onset of one or more symptoms of a disorder in a subject resulting from the administration of a prophylactic or therapeutic agent.

As used herein, the term “in combination” refers to the use of more than one prophylactic and/or therapeutic agents. The use of the term “in combination” does not restrict the order in which prophylactic and/or therapeutic agents are administered to a subject with a disorder. A first prophylactic or therapeutic agent can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject which had, has, or is susceptible to a disorder. The prophylactic or therapeutic agents are administered to a subject in a sequence and within a time interval such that the agent of the invention can act together with the other agent to provide an increased benefit than if they were administered otherwise. Any additional prophylactic or therapeutic agent can be administered in any order with the other additional prophylactic or therapeutic agents.

### 5. BRIEF DESCRIPTION OF THE DRAWINGS

FIG. 1. Apoptosis and cell cycle analysis of A375 cells cultured in normoxia or hypoxia for 24 hours. (A), Representative flow cytometric analysis of cell cycle using propidium iodide for DNA staining: shown is the pattern of A375 cells being in apoptosis and in G₀/G₁, S and G₂/M phase during normoxia and hypoxia. Apoptotic cells (Apo) with sub-diploid DNA content are reported. (B), The quantitative analysis of sub-diploid and of cells in G₀/G₁, S and G₂/M phase is given in the graph. Plots are mean±S.E. values (n=3). *P<0.01 compared with hypoxia.

FIG. 2. Induction of HIF-1 expression by hypoxia. A375 cells were cultured in normoxia for 4 hours (lane normoxia) or under hypoxic conditions for 2, 3, 4, 8, 16 and 24 hours (lanes 2-7). Whole cellular protein extracts were prepared and subjected to immunoblot assay using an anti-HIF-1α monoclonal antibody. The blot was then stripped and used to determine HIF-1β expression using an anti-HIF-1β monoclonal antibody. Tubulin shows equal loading protein.

FIG. 3. Induction of HIF-1α expression by adenosine. (A), A375 cells were cultured in normoxia for 4 hours (lane normoxia). A375 cells were treated without (lane 1) or with adenosine 10 nM (lane 2), 100 nM (lane 3), 1 μM (lane 4), 10 μM (lane 5) and 100 μM (lane 6) in hypoxia for 4 hours. Cellular extracts were prepared and subjected to immunoblot assay using an anti-HIF-1α monoclonal antibody. The blot was then stripped and used to determine HIF-1β expression using an anti-HIF-1β monoclonal antibody. Tubulin shows equal loading protein. (B), Typical dose response curve of A375 cells exposed to adenosine in hypoxia is shown. The HIF-1α immunoblot signals were quantified using molecular analyst /PC densitometry software (Bio-Rad). The mean densitometry data from 12 independent experiments (one of which is shown in Panel A) were normalized to the result obtained in cells in the absence of adenosine (control). Plots are mean±S.E. values (n=12). (C), Effect of A₁, A_{2A}, A_{2B} and A₃ adenosine receptor antagonists. A375 cells were treated without (lane 1, control) or with adenosine 100 μM (lanes 2-6) and exposed to the A₁ receptor antagonist DPCPX 100 nM (lane 3), or A_{2A} receptor antagonist SCH 58261 100 nM (lane 4), or A_{2B} receptor antagonist MRE 2029F20 100 nM (lane 5), or A₃ receptor antagonist MRE 3008F20 100 nM (lane 6) in hypoxia for 4 hours. Cellular extracts were prepared and subjected to immunoblot assay using an anti-HIF-1α monoclonal antibody. The blot was then stripped and used to determine HIF-1β expression using an anti-HIF-1β monoclonal antibody. Tubulin shows equal loading protein. (D), The immunoblot signals were quantified using molecular analyst/PC densitometry software (Bio-Rad). The mean densitometry data from 5 independent experiments (one of which is shown in Panel C) were normalized to the result obtained in cells in the absence of adenosine (control). Plots are mean±S.E. values (n=5). *P<0.01 compared with the control.

FIG. 4. Induction of HIF-1 expression by A₃ receptor stimulation: time course. (A), A375 cells were cultured in normoxia for 4 hours (lane normoxia) or under hypoxic conditions in the absence (−) or in the presence (+) of the A₃ receptor agonist Cl-IB-MECA (100 nM) for 2, 4, 8, 16 and 24 hours. Whole cellular protein extracts were prepared and subjected to immunoblot assay using an anti-HIF-1α monoclonal antibody. The blot was then stripped and used to determine HIF-1β expression using an anti-HIF-1β monoclonal antibody. Tubulin shows equal loading protein. (B), The immunoblot signals were quantified using molecular analyst/PC densitometry software (Bio-Rad). The mean densitometry data from 3 independent experiments (one of which is shown in Panel A) were normalized to the result obtained in cells in the absence of Cl-IB-MECA after 4 hours of hypoxia (control). Plots are mean±S.E. values (n=3). *P<0.01 compared with the control.

FIG. 5. Induction of HIF-1α expression by A₃ receptor stimulation: dose response. (A), A375 cells were treated without (lane 1) or with Cl-IB-MECA 0.11 nM (lane 2), 1 nM (lane 3), 10 nM (lane 4) 100 nM (lane 5) and 1 μM (lane 6) in normoxia and in hypoxia for 4 hours. Cellular extracts were prepared and subjected to immunoblot assay using an anti-HIF-1α monoclonal antibody. The blot was then stripped and used to determine HIF-1β expression using an anti-HIF-1β monoclonal antibody. (B), Typical dose response curve of A375 cells exposed to adenosine in hypoxia is shown. The immunoblot signals were quantified using molecular analyst /PC densitometry software (Bio-Rad). The mean densitometry data from 12 independent experiments (one of which is shown in Panel A) were normalized to the result obtained in cells in the absence of Cl-IB-MECA (control). Plots are mean±S.E. values (n=12).

FIG. 6. Effect of A₃ receptor antagonist MRE 3008F20. (A), A375 cells were treated in hypoxia for 4 hours without (−) or with (+) Cl-IB-MECA 10 nM, MRE 3008F20 10 nM (lanes 3, 4) and MRE 3008F20 100 nM (lanes 5, 6). Cellular extracts were prepared and subjected to immunoblot assay using an anti-HIF-1α monoclonal antibody. The blot was then stripped and used to determine HIF-1β expression using an anti-HIF-1β monoclonal antibody. (B), The immunoblot signals were quantified using molecular analyst /PC densitometry software (Bio-Rad). The mean densitometry data from independent experiments (one of which is shown in Panel A) were normalized to the result obtained in hypoxic cells in the absence of Cl-IB-MECA (control, lane 1). Plots are mean±S.E. values (n=3); *P<0.01 compared with the control. (C), A375 cells were treated in hypoxia for 4 hours without (lane 1) or with Cl-IB-MECA 10 nM (lanes 2-6) and MRE 3008F20 0.3 nM (lane 3), 1 nM (lane 4), 3 nM (lane 5) and 10 nM (lane 6). Cellular extracts were prepared and subjected to immunoblot assay using an anti-HIF-1α monoclonal antibody. The blot was then stripped and used to determine HIF-1β expression using an anti-HIF-IP monoclonal antibody. (D), Typical dose response curve of A375 cells exposed to MRE 3008F20 in hypoxia is shown. The immunoblot signals were quantified using molecular analyst /PC densitometry software (Bio-Rad). The mean densitometry data from 3 independent experiments (one of which is shown in Panel C) were normalized to the result obtained in cells in the absence of Cl-IB-MECA (control). Plots are mean±S.E. values (n=3).

FIG. 7. A₃ receptor expression silencing by siRNA transfection. (A), Analysis of siRNA transfection efficiency in A375 cells. Representative flow chromatograms of siRNA-FITC accumulation (gray filled area) in A375 cells transfected with siRNA-FITC. Unfilled area shows A375 cells transfected with RNAiFect™ Transfection reagent without siRNA_{A3}. Fluorescence was quantified by flow cytometry 5 hours post-transfection. (B), Relative A₃ receptor mRNA quantification, related to β-actin mRNA, by Real-Time RT-PCR. A375 cells were transfected by RNAiFect™ Transfection reagent or siRNA_{A3} and cultured for 24, 48 and 72 hours. Plots are mean±S.E. values (n=3); *P<0.01 compared with the control. (C), Western blot analysis using an anti-A₃ receptor polyclonal antibody of protein extracts from A375 cells treated by RNAiFect™ Transfection reagent (control) or siRNA_{A3} and cultured for 24, 48 and 72 hours in normoxia. Tubulin shows equal loading protein. (D), Densitometric quantification of A₃ receptor Western blot; plots are mean±S.E. values (n=5); *P<0.01 compared with the control. (E), Western blot analysis using an anti-HIF-1α monoclonal antibody of protein extracts from A375 cells treated by control-siRNA (−) or siRNA_{A3} (+) for 72 hours and cultured with (+) or without (−) Cl-IB-MECA 100 nM for 4 hours in hypoxia. Tubulin shows equal loading protein.

FIG. 8. A₃ receptor stimulation induces HIF-1α accumulation in various human cell lines under hypoxia. NCTC 2544 keratinocytes, U87MG glioblastoma, U2OS osteosarcoma human cells were treated without (−) or with (+) Cl-IB-MECA 100 nM in hypoxia for 4 hours. Cellular extracts were prepared and subjected to immunoblot assay using an anti-HIF-1α monoclonal antibody. The blot was then stripped and used to determine HIF-1β expression using an anti-HIF-1β monoclonal antibody.

FIG. 9. A₃ receptor stimulation induces HIF-1α accumulation through a transcription-independent pathway. (A), A375 cells were pretreated with actinomycin D (10 μg/ml) for 30 minutes and then exposed to hypoxia. HIF-1α accumulation was induced by the exposure of A375 cells to Cl-IB-MECA 100 nM (+) for 4 hours in hypoxia in the absence (lane 2) or in the presence (lane 4) of actinomycin D. Cellular extracts were prepared and subjected to immunoblot assay using an anti-HIF-1α monoclonal antibody. Tubulin shows equal loading protein. (B), The immunoblot signals were quantified using molecular analyst/PC densitometry software (Bio-Rad). The mean densitometry data from independent experiments (one of which is shown in Panel A) were normalized to the result obtained in cells in the absence of Cl-IB-MECA after 4 hours of hypoxia (control=lane 1). Plots are mean±S.E. values (n=3); *P<0.01 compared with the control.

FIG. 10. Induction of HIF-1α accumulation by A₃ receptor stimulation in normoxia. (A), A375 cells were exposed to 100 μM CoCl₂ alone (lane 1) or in the presence of Cl-IB-MECA 1 nM (lane 2), 10 nM (lane 3), 100 nM (lane 4), 1 μM (lane 5) and 10 μM (lane 6) in normoxia for 4 hours. Cellular extracts were prepared and subjected to immunoblot assay using an anti-HIF-1α monoclonal antibody. The blot was then stripped and used to determine HIF-1β expression using an anti-HIF-1β monoclonal antibody. (B), A375 cells were exposed to 100 μM CoCl₂ for 4 hours in normoxia. HIF-1α accumulation was induced by the exposure of A375 cells to Cl-IB-MECA 100 nM (+) for 4 (lanes 1 and 4) or 6 hours (lanes 5 and 6) in the absence (lanes 1 and 2) or in the presence (lanes 3-6) of cycloheximide (1 μM). Cellular extracts were prepared and subjected to immunoblot assay using an anti-HIF-1α monoclonal antibody. Tubulin shows equal loading protein.

FIG. 11. A₃ receptor activation does not affect HIF-1α degradation in normoxia. (A), A375 cells were incubated in hypoxia in the absence (lanes 1 to 4) or in the presence of Cl-IB-MECA 100 nM (lanes 5 to 8). After 4 hours, melanoma cells were exposed to normoxia and a time-course of HIF-1α disappearance was performed at 0, 5, 10 and 15 minutes. Cellular extracts were prepared and subjected to immunoblot assay using an anti-HIF-1α monoclonal antibody. Tubulin shows equal loading protein. (B), The immunoblot signals were quantified using molecular analyst /PC densitometry software (Bio-Rad). The mean densitometry data from 3 independent experiments (one of which is shown in Panel A) were normalized to the result obtained in cells at time 0 (control). The fraction of HIF-1α remaining is indicated.

FIG. 12. Role of p38, p44 and p42 MAPKs in A₃ signalling. (A), A375 cells were pretreated with or without SB202190 (1 and 10 μM) or U0126 (10 and 30 μM) and then exposed to Cl-IB-MECA 100 nM (+) or to drug vehicle (−) for 4 hours in hypoxia. Cellular extracts were prepared and subjected to immunoblot assay using an anti-HIF-1α monoclonal antibody. Tubulin shows equal loading protein. (B), A₃ stimulation via Cl-IB-MECA induces p44/p42 activation after 4 hours hypoxia in A375 cells. Cl-IB-MECA 0 (lane C), 10 (lane 1), 100 (lane 2), 500 (lane 3) and 1000 (lane 4) nM was added to A375 cells. After 4 hours cells were harvested and subjected to immunoblot assay using antibodies specific for phosphorylated (Thr183/Tyr185) or total p44/p42 MAPKs. (C), The immunoblot signals were quantified using molecular analyst /PC densitometry software (Bio-Rad). Densitometric analysis of p44 and p42 phosphorylated isoforms is reported. The mean densitometry data from 3 independent experiments (one of which is shown in Panel B) were normalized to the results obtained in cells in the absence of Cl-IB-MECA (lane C). Plots are mean±S.E. values (n=3); *P<0.01 compared with the control (lane C). (D), A₃ stimulation via Cl-IB-MECA induces p38 activation after 4 hours hypoxia in A375 cells. Cl-IB-MECA 0 (lane C), 10 (lane 1), 100 (lane 2), 500 (lane 3) and 1000 (lane 4) nM was added to A375 cells. After 4 hours cells were harvested and subjected to immunoblot assay using antibodies specific for phosphorylated (Thr180/Tyr182) or total p38 MAPKs. (E), The immunoblot signals were quantified using molecular analyst /PC densitometry software (Bio-Rad). Densitometric analysis of p38 phosphorylated isoforms is reported. The mean densitometry data from 3 independent experiments (one of which is shown in Panel D) were normalized to the result obtained in cells in the absence of Cl-IB-MECA (lane C). Plots are mean±S.E. values (n=3); *P<0.01 compared with the control (lane C).

### 6. DETAILED DESCRIPTION OF THE INVENTION

The present invention relates to methods for the treatment, prevention, and/or management of diseases or disorders associated with a reduced expression of HIF-1α and/or decreased HIF-1α activity (e.g., ischemic cardiac disorders) by using A₃ receptor agonists alone or in combination with A₁ receptor agonists. Although not intending to be bound by a particular mechanism of action the A₃ receptor agonists of the invention up-regulate HIF-1α expression and thus promote angiogenesis and reversal of ischemic damage as a result of low level of HIF-1α expression and/or activity. In most preferred embodiments, the methods of the invention relate to treatment, prevention, and/or management of diseases or disorders associated with underexpression of HIF-1α and/or decreased HIF-1α activity by using A₃ receptor agonists alone.

The compounds and methods of the invention are particularly useful for increasing vasogenesis or angiogenesis to treat diseases or conditions associated with insufficient vascularization, or an injury to vessels. For example, the A₃ receptor agonists may be administered to individuals having undergone surgery, particularly vessel or cardiac surgery, to improve the rate of vessel repair. In a second example, the A₃ receptor agonists may be used to treat individuals having insufficient peripheral blood flow, such as individual having a non-healing wound, or Reynaud's disease. Thus, in another embodiment, the invention provides a method of treating an individual, wherein said individual has a condition or disease associated with insufficient angiogenesis or vasogenesis, comprising administering to said individual an amount of an A₃ receptor agonist that detectably increases angiogenesis or vasogenesis, said agonist administered in an amount sufficient to increase said angiogenesis or vasogenesis

The A₃ receptor agonists used in the methods and compositions of the invention are particularly useful for reducing ischemia of an organ in a mammal at risk for ischemia. The methods of the invention encompass administering to said mammal an effective amount of a pharmaceutical composition comprising a selective A₃ receptor agonist. Ischemia is a deficiency of oxygenated blood. The deficiency of blood may, for example, be caused by functional constriction or obstruction of a blood vessel. Several organs are subject to ischemia including, but not limited to, the heart, brain, kidney, and intestines. The methods of the invention are effective in reducing ischemia in one or more organs.

In some embodiments, the invention encompasses treating and/or preventing ischemic heart disease using one or more A₃ receptor agonists either alone or in combination with other therapeutic and/or prophylactic agents. Although not intending to be bound by a particular mechanism of action ischemia is often caused by a reduction in coronary blood flow relative to myocardial demand. The reduction in blood flow may result from a variety of reasons, and typically occurs as a result of atherosclerosis. The methods of the invention are effective in reducing ischemic related impaired blood flow or other ischemic related tissue or organ damage including damage to the heart muscle, cardiac arrhythmias, angina, myocardial infarction, congestive heart failure, and sudden cardiac death. Ischemia may be assessed by any method known to those skilled in the art. An assessment of ischemic damage may be made, for example, by measuring the infarct (scar) size of the organ.

The methods of the invention are directed to methods of reducing tissue damage (e.g., substantially preventing tissue damage, inducing tissue protection) resulting from ischemia or hypoxia comprising administering to a mammal in need of such treatment a therapeutically effective amount of an A₃ receptor agonist, a pharmaceutically acceptable salt of said compound. Preferred ischemic or hypoxic tissues taken individually or as a group are where the ischemic or hypoxic tissue is cardiac, brain, liver, kidney, lung, gut, skeletal muscle, spleen, pancreas, nerve, spinal cord, retina tissue, the vasculature, or intestinal tissue. An especially preferred ischemic or hypoxic tissue is cardiac tissue. It is especially preferred that the compounds are administered to prevent perioperative myocardial ischemic injury. Preferably, the A₃ receptor agonists are administered prophylactically. The ischemic or hypoxic damage may occur during organ transplantation. Preferably, the A₃ receptor agonists are administered prior to, during or shortly after, cardiac surgery or non-cardiac surgery.

Another aspect of this invention is directed to methods of reducing myocardial tissue damage (e.g., substantially preventing tissue damage, inducing tissue protection) during surgery (e.g., coronary artery bypass grafting (CABG) surgeries, vascular surgeries, percutaneous transluminal coronary angioplasty (PTCA) or any percutaneous transluminal coronary intervention (PTCI), organ transplantation, or other non-cardiac surgeries) comprising administering to a mammal a therapeutically effective amount of a compound of an A₃ receptor agonist of the invention.

In another aspect, the invention relates to a method of preserving an organ from a mammal, comprising storing the organ in a solution comprising an effective amount of an adenosine A₃ receptor agonist. For example, an effective amount of the A₃ agonist may contained in an organ storage solution along with one or more buffer systems.

The methods and compositions of the invention comprising A₃ receptor agonists are particularly useful when the levels of HIF-1α expression and/or activity are reduced below the standard or background level, as determined using methods known to those skilled in the art and dislosed herein.

As used herein, “restoration” of a measured level of HIF-1α to a standard level means that the amount or concentration of HIF-1α in a sample or subject is lower in a subject or sample relative to the standard as detected by any method now known in the art or to be developed in the future for measuring HIF-1α levels and the methods of the invention allow the level to return to the background or standard level. Such a restoration may include, but is not limited to a restoration of HIF-1α level to a level which is about a 10%, about a 20%, about a 40%, about an 80%, about a 2-fold, about a 4-fold, about an 10-fold, about a 20-fold, about a 50-fold, about a 100-fold, about a 2 to 20 fold, 2 to 50 fold, 2 to 100 fold, 20 to 50 fold, 20 to 100 fold within the standard level. The term “about” as used herein, refers to levels of elevation of the standard numerical value plus or minus 10% of the numerical value.

The term “standard level” or “background level” as used herein refers to a baseline amount of an HIF-1α level as determined in one or more normal subjects, i.e., a subject with no known history of past or current diseases or disorders. For example, a baseline may be obtained from at least one subject and preferably is obtained from an average of subjects (e.g., n=2 to 100 or more), wherein the subject or subjects have no prior history of diseases or disorders, especially no prior history of diseases associated with HIF-1α.

In the present invention, the measurement of an HIF-1α level may be carried out using an HIF-1α probe or a HIF-1α activity assay (see Section 5.3.4). As used herein, reference to measuring a level of HIF-1α in a method of the invention relates to any proxy for HIF-1α levels. For example, such levels may include, but are not limited to, the abundance of HIF-1α nucleic acid or amino acid sequences in a sample from a subject. A level of HIF-1α may correspond to the abundance of full-length HIF-1α protein. Alternatively, a level of HIF-1α, may correspond to abundance of a fragment, analog or derivative of HIF-1α protein. A level of HIF-1α can be determined by measuring the abundance of nucleic acids (or sequences complementary thereto) that corresponds to all or fragments of HIF-1α. In a preferred embodiment, the abundance of mRNA encoding HIF-1α is measured.

As used herein, a probe with which the amount or concentration of HIF-1α can be determined, includes but is not limited to an antibody, an antigen, a nucleic acid, a protein, or a small molecule. In a specific embodiment, the probe is the HIF-1α protein or a fragment thereof. In another embodiment, the probe is an antibody that immunospecifically binds to HIF-1α, such as e.g., a monoclonal antibody or a binding fragment thereof.

In a specific embodiment, measuring a level of HIF-1α comprises testing at least one aliquot of the sample, said step of testing comprising: (a) contacting the aliquot with an antibody or a fragment thereof that is immunospecific for HIF-1α, and (b) detecting whether and how much binding has occurred between the antibody or a fragment thereof and at least one species of HIF-1α in the aliquot. In yet another specific embodiment, measuring a level of HIF-1α comprises testing at least one aliquot, said step of testing comprising: (a) contacting the aliquot with a nucleic acid probe that is hybridizable to HIF-1α mRNA, and (b) detecting whether and how much hybridization has occurred between the nucleic acid probe and at least one species of HIF-1α mRNA in the aliquot. In both embodiments measuring a level of HIF-1α involves quantitating the amount of complex formation. For example the amount of complex formation between the antibody or a fragment thereof and at least one species of HIF-1α in the aliquot would correlate with the amount of at least one species of HIF-1α in the aliquot of the sample.

In a further specific embodiment, the antibody or other probe is labeled with a detectable marker. In yet another specific embodiment, the detectable marker is a chemiluminescent, enzymatic, fluorescent, or radioactive label.

The therapeutic methods of the invention comprising administering a therapeutically effective amount of an A₃ receptor agonist of the invention to improve the therapeutic efficacy of diseases or disorders associated with a reduced expression of HIF-1α and/or decreased HIF-1α activity (e.g., ischemic disorders) relative to the traditional modes of such therapies.

Preferably the methods of the invention increase the HIF-1α level to the standard level within at least 1 day, 1 week, one month, 2 months, at least 4 months, at least 6 months of the therapeutic regime. In a most preferred embodiment, the methods of the invention result in a complete restoration of HIF-1α level to the background level. The invention encompasses restoration of the HIF-1α level to a level which is within about about 10%, about 20%, about 30%, about 40%, about 50% of the background level.

In a preferred specific embodiment, the invention encompasses a method for treatment, prevention and/or management of diseases or disorders associated with a reduced expression of HIF-1α and/or decreased HIF-1α activity (e.g., ischemic disorders) comprising administering a therapeutically and/or prophylactically effective amount of an A₃ receptor agonist compound as disclosed herein.

The A₃ receptor agonists for use in the compositions and methods of the invention, either alone or in combination with other therapeutic and prophylactic agents (including A₁ receptor agonists) are particularly useful for reducing hypoxia or ischemia-related tissue damage in a subject having or at risk of such damage. The invention relates to methods and compositions for the treatment, prevention and/or management of a HIF-1α mediated disease or disorder by using an A₃ receptor agonist of the invention. The methods of the invention contemplate administering a therapeutically and/or prophylactically effective amount of the A₃ receptor agonists alone or in combination with other agents. The A₃ receptor agonists for use in the compositions and methods of the invention are particularly useful for the treatment of HIF-1α associated disease or disorder including without limitation, ischemic cardiovascular disorders (e.g., myocardial ischemia, cerebral ischemia, retinal ischemia), pulmonary hypertension, pregnancy disorders, (e.g., preeclampsia, intrauterine growth retardation), any surgical procedures wherein the blood supply needs to be shut off, or any other disorder with impaired blood flow.

In specific embodiments, the methods of the invention may be used to treat a peripheral arterial disease. For instance, in some embodiments the peripheral arterial disease is gangrene. deep vein thrombosis or vascular insufficiency.

In other specific embodiments, the methods of the invention may be used to treat a disorder associated with impaired cerebral circulation such as stroke or multi-infarct dementia.

Although not intending to be bound by a particular mechanism of action the agonists of the invention are therapeutically effective by increasing the level and/or activity of HIF-1α or HIF-1α related activity which will promote angiogenesis. Overexpression of HIF-1α leads to dimerization with endogenous HIF-1β and activation of hypoxia inducible genes relevant to angiogenesis including but not limited to vascular endothelial growth factor.

The invention encompasses compounds which are A₃ receptor agonists for use in the methods of the invention. Examples of such compounds are disclosed in U.S. Patent Application Publication Nos. 20040204481 A1; 20040198693 A1; 20040121978 A1; 20040116376 A1; 20040106572 A1; 20030166605 A1; 20030143282 A1; 20030078232 A1; 20020165197; 20020115635 and U.S. Pat. Nos. 6,586,413; 6,448,253; 6,407,236; 6,358,964; 6,329,349; 6,211,165; 5,573,772; and 5,443,836; all of which are incorporated herein by reference in their entireties.

The present invention encompasses therapies which involve administering one or more A₃ receptor agonists, to an animal, preferably a mammal, and most preferably a human, for preventing, treating, or ameliorating symptoms associated with a disease or disorder associated with hypoxia-inducible factor 1-α (HIF-1α).

The invention further provides a pharmaceutical composition comprising a therapeutically or prophylactically effective amount of an A₃ receptor agonist and a pharmaceutically acceptable carrier The compounds can be used in a pharmaceutical formulation that also includes an adenosine A₁, A_{2B}, or A_{2A} receptor agonist and one or more excipients.

The invention also encompasses a method for determining the prognosis of a a disease or disorder associated with under-expression of HIF-1α and/or decreased HIF-1α activity in a subject. Preferably, the subject is human, and most preferably the subject has been previously treated with a therapy regimen. The invention encompasses measuring at least a level of HIF-1 α in a subject to determine if the subject is in need of the therapeutic and or prophylcatic methods of the invention. The invention encompasses measuring a level of aHIF-1 α in a sample obtained from the subject and comparing the level measured to a standard level, wherein reduction of the measured level of HIF-1 α relative to the standard level indicates that the subject is at an increased risk for progression of the disease or disorder, e.g., ischemic disorder.

6.1 Prophylactic and Therapeutic Methods

The present invention relates to methods for the treatment, prevention, and/or management of diseases or disorders associated with a reduced expression of HIF-1α and/or decreased HIF-1α activity (e.g., ischemic disorders) by using A₃ receptor agonists alone or in combination with A₁, A_{2B} or A_{2A} receptor agonists. In most preferred embodiments, the methods of the invention relate to treatment, prevention, and/or management of diseases or disorders associated with a reduced expression of HIF-1α and/or decreased HIF-1α activity by using A₃ receptor agonists alone.

The invention provides methods for treatment of HIF-1-mediated disorders, including hypoxia- or ischemia-related tissue damage, which are improved or ameliorated by modulation of HIF-1 expression or activity. The relevant clinical conditions treated by the methods and compositions of the invention include ischemia due to a disease of the cerebral, coronary, or peripheral circulation. One therapeutic goal of the invention is to promote angiogenesis in the ischemic tissue by enhancing HIF-1α expression and/or activity. Although not intending to be bound by a particular mechanism of action, such an enhancement may result in dimerization of HIF-1α with endogenous HIF-1β, binding to specific DNA sequences, and activation transcription of hypoxia-inducible genes relevant to angiogenesis, such as, but not limited to, the gene encoding vascular endothelial growth factor (VEGF), a known HIF-1 target gene.

The methods of the invention are directed to methods of reducing tissue damage (e.g., substantially preventing tissue damage, inducing tissue protection) resulting from ischemia or hypoxia comprising administering to a mammal in need of such treatment a therapeutically effective amount of an A₃ receptor agonist, a pharmaceutically acceptable salt of said compound. Preferred ischemic or hypoxic tissues that benefit from the methods and compositions of the invention include without limitation cardiac, brain, liver, kidney, lung, gut, skeletal muscle, spleen, pancreas, nerve, spinal cord, retina tissue, the vasculature, or intestinal tissue.

Another aspect of this invention is directed to chronic methods of reducing myocardial tissue damage (e.g., substantially preventing tissue damage, inducing tissue protection) in a patient with diagnosed coronary heart disease (e.g., previous myocardial infarction or unstable angina) or patients who are at high risk for myocardial infarction (e.g., age>65 and two or more risk factors for coronary heart disease) comprising administering to a mammal a therapeutically effective amount of a compound as disclosed herein.

The invention relates to methods and compositions for the treatment, prevention and/or management of ischemic or hypoxic damage, cardiovascular diseases, arteriosclerosis, arrhythmia, angina pectoris, cardiac hypertrophy, renal diseases, restenosis, septic shock and other inflammatory diseases, and cerebral ischemic disorders.

The invention encompasses adminstration of one or more compounds of the invention for minimizing ischemic damage and/or reperfusion injury to heart tissue during cardiac surgery, for example, where the heart is removed from the body and then re-implanted into the same body, as well as cardiac transplantation, where the heart is removed from one body and transplanted into another body. Such methods are disclosed in U.S. Publication No. 2003/0166605 to Leung et al. which is incorporated herein by reference in its entirety. Prior to removing the heart from the body, adenosine A₃ receptor agonists can be administered to the patient in a manner which provides cardioprotection to the heart.

In other embodiments, the invention relates to methods and compositions for the treatment, prevention and/or management of a HIF-1α mediated disease or disorder by using an A₃ receptor agonist. The methods of the invention contemplate administering a therapeutically and/or prophylactically effective amount of the A₃ receptor agonists alone or in combination with other therapeutic and/or prophylactic agents. The A₃ receptor agonists are particularly useful for the treatment of HIF-1α associated disease or disorder including without limitation, ischemic cardiovascular disorders (e.g., myocardial ischemia, cerebral ischemia, retinal ischemia), pulmonary hypertension, pregnancy disorders, (e.g., preeclampsia, intrauterine growth retardation), any surgical procedure wherein the blood supply needs to be shut off, or any other disorder with impaired blood flow.

The A₃ receptor agonists to be used in the compositions and methods of the invention function as a prophylactic and/or therapeutic agents of a disease or disorder and can be administered to an animal, preferably a mammal, and most preferably a human, to treat, prevent or ameliorate one or more symptoms associated with the disease or disorder. The subject is preferably a mammal such as non-primate (e.g., cows, pigs, horses, cats, dogs, rats, etc.) and a primate (e.g., monkey, such as a cynomolgous monkey and a human). In a preferred embodiment, the subject is a human. Compounds of the invention can be administered in combination with one or more other prophylactic and/or therapeutic agents useful in the treatment, prevention or management of a HIF-1α-mediated disorders, including hypoxia- or ischemia-related tissue damage, which are improved or ameliorated by modulation of HIF-1 expression or activities.

The A₃ receptor agonists (alone or in combination with A₁, A_{2A} and A_{2B} receptor agonists) required to be effective will, of course, vary with the individual mammal being treated and is ultimately at the discretion of the medical or veterinary practitioner. The factors to be considered include the condition being treated, the route of administration, the nature of the formulation, the mammal's body weight, surface area, age and general condition, and the particular compound to be administered. However, a suitable effective dose is in the range of about 0.1 μg/kg to about 100 mg/kg, about 0.1 μg/kg to about 500 mg/kg, about 0.1 μg/kg to about 1 g/kg, about 100 ug/kg to about 500 mg/kg, about 100 ug/kg to about 1 g/kg, about 1 mg/kg to about 100 mg/kg, about 1 mg/kg to about 500 mg/kg, about 1 mg/kg to about 1 g/kg of the patient's body weight. The total daily dose may be given as a single dose, multiple doses, e.g., two to six times per day, or by intravenous infusion for a selected duration. Dosages above or below the range cited above are within the scope of the present invention and may be administered to the individual patient if desired and necessary.

The methods and compositions of the invention comprise the administration of one or more compounds of the invention to subjects/patients suffering from or expected to suffer from a disease or disorder.

The invention relates to methods and compositions for the treatment, prevention and/or management of a HIF-1α mediated disease or disorder by using an A₃ receptor agonist of the invention. The methods of the invention are particularly effective in reducing cellular damage related to an ischemic condition and for treatment of disorders related to ischemia-related cellular damage or death. Such disorders include, but are not limited to ischemia, glaucoma and other neurodegenerative diseases, as well as cardiac injury associated with myocardial infarction. While such disorders are usually characterized by apoptotic cell death, apoptosis or necrosis may or may not be involved.

The methods of the invention contemplate administering a therapeutically and/or prophylactically effective amount of the A₃ receptor agonists alone or in combination with other therapeutic and/or prophylactic agents. The agonists of the invention including the A₃ receptor agonists are particularly useful for the treatment of HIF-1α associated disease or disorder including without limitation, ischemic cardiovascular disorders (e.g., myocardial ischemia, cerebral ischemia, retinal ischemia, cardiomyopathy, congestive heart failure, coronary artery disease, hypertension, ischemia/reperfusion, restenosis and vascular stenosis), pulmonary hypertension, pregnancy disorders, (e.g., preeclampsia, intrauterine growth retardation), ischemia; ischemic conditions associated with surgery or traumatic injury, any surgical procedures wherein the blood supply needs to be shut off, or any other disorder with impaired blood flow. Although not intending to be bound by a particular mechanism of action the agonists of the invention are therapeutically effective by increasing the level and/or activity of HIF-1α or HIF-1α related activity which will promote angiogenesis. Overexpression of HIF-1α leads to dimerization with endogenous HIF-1β and activation of hypoxia inducible genes relevant to angiogenesis including but not limited to vascular endothelial growth factor.

A₃ receptor agonists may be used to treat any ischemic tissue, e.g., a tissue having a deficiency in blood as the result of an ischemic disease. Tissues deprived of blood and oxygen undergo ischemic necrosis or infarction with possible irreversible organ damage. A₃ receptor agonists of the invention are particularly effective in reducing, preventing, or treating such conditions. Such tissues can include, for example, muscle, brain, kidney and lung. Ischemic diseases include, for example, cerebrovascular ischemia, renal ischemia, pulmonary ischemia, limb ischemia, ischemic cardiomyopathy and myocardial ischemia.

The invention provides treatment methods for reducing cellular damage related to an ischemic condition in a subject, preferably a human subject by administration of one or more A₃ receptor agonists. The ischemic condition may be due to an interruption in circulation, such as that caused by cardiac failure, or other condition leading to global loss of blood supply to a tissue or organ, or due to localized interruptions in blood flow, such as those due to cerebral hemorrhaging, or localized thrombotic events. Alternatively, the damage may be to myocardial tissue, as resulting from decreased perfusion of the coronary arteries (heart attack). The A₃ receptor agonists are able to modulate the cell death associated with ischemic injury.

In one specific embodiment, the invention relates to treatment of cardiac ishcemia using one or more A₃ receptor agonists. Cardiac ischemia, is a condition characterized by a reduced blood flow to the heart. It is usually the result of the build-up of plaque in the coronary arteries. In many cases, ischemia has no symptoms (silent ischemia). Minor episodes of cardiac ischemia tend to cause little long-term damage to the heart, but these episodes can sometimes cause serious effects in some patients: They can cause abnormal heart rhythms (arryhtmmias), which can lead to either syncope (fainting) or cardiac arrest (the abrupt inability of the heart to pump blood) and sudden cardiac death. Severe or lengthy episodes can trigger a heart attack. The collective effects of minor episodes of cardiac ischemia can potentially lead to a weakening of the heart muscle (cardiomyopathy). The A₃ receptor agonists may be combined with other therapeutic or prophylactic methods known in the art for the treatment of cardiac ischemia including but not limited to beta blockers, calcium channel blockers and nitrates. Although not intending to be bound by a particular mechanism of action since cardiac ischemia is due to the heart not getting enough oxygen, these drugs reduce the heart's need for oxygen, for example by slowing the heart rate, reduceing blood pressure and relaxing the blood vessels. Other agents that may be used include aspirin and other anti-platelet agents to decrease the chance of a blood clot forming in the narrowed artery.

In one specific embodiment, the invention relates to treatment of cerebral ishcemia using one or more A₃ receptor agonists. Although not intending to be bound by a particular mechanism of action, cerebral ischemia results from decreased blood and oxygen flow implicating one or more of the blood vessels of the brain. In cerebral ischemia, the individual suffers a stroke with sudden development of a focal neurologic deficit and, in most cases, some degree of brain damage. The decreased blood flow may be due to, for example, an occlusion such as a thrombus or embolus, vessel rupture, sudden fall in blood pressure, change in the vessel lumen diameter due to atherosclerosis, trauma, aneurysm, developmental malformation, altered permeability of the vessel wall or increased viscosity or other quality of the blood. Decreased blood flow may also be due to failure of the systemic circulation and severe prolonged hypotension. Ischemic necrosis of the spinal cord may result in sensory or motor symptoms or both that can be referred to cervical, thoracic or lumbar levels of the spine.

In another specific embodiment, the invention relates to treatment of ischemic heart disease using one or more A₃ receptor agonists. Ischemic heart disease results from an imbalance between myocardial oxygen supply and demand. In ischemic heart disease, the individual suffers angina pectoris, acute myocardial infarction or sudden death. The imbalance may be caused by, for example, atherosclerotic obstruction of one or more large coronary arteries, nonatheromatous coronary obstructive lesions such as embolism, coronary ostial stenosis associated with luetic aortitis, coronary artery spasm, congenital abnormalities of the coronary circulation, increased myocardial oxygen demands exceeding the normal supply capabilities as in severe myocardial hypertrophy, reduction in the oxygen carrying capacity of the blood such as anemia, or as a consequence of inadequate cardiac perfusion pressure due to hypotension from any cause.

The present invention provides methods to treat/or prevent cardiovascular tissue damage resulting from cardiac ischemia or reperfusion injury. Reperfusion injury, for instance, occurs at the termination of cardiac bypass procedures or during cardiac arrest when the heart, once prevented from receiving blood, begins to reperfuse and these methods involve administration of the compounds and compositions of the present invention preferably prior to, or immediately subsequent to reperfusion, such that reperfusion injury is prevented, treated or reduced. The present invention also provides methods of preventing and/or treating vascular stroke, cardiovascular disorders.

Other ischemic disorders that may be treated, prevented and/or managed using the methods and compositions of the invention include myocardial ischemia, cerbral ischemia, retinal ischemia.

Although not intending to be bound by a particular mechanism of action, A₃ receptor agonists pharmacologically mimic the cardioprotective effects of ischemic preconditioning by activating adenosine A₃ receptors and hence are useful as therapeutic or prophylactic agents for diseases caused or aggravated by ischemia or hypoxia, or ischemia/reperfusion for example, cardiovascular diseases [e.g., arteriosclerosis, arrhythmia (e.g. ischemic arrhythmia, arrhythmia due to myocardial infarction, myocardial stunning, myocardial dysfunction, arrhythmia after thrombolysis, etc.), angina pectoris, cardiac hypertrophy, myocardial infarction, heart failure (e.g. congestive heart failure, acute heart failure, cardiac hypertrophy, etc.), restenosis, shock (e.g. hemorrhagic shock, endotoxin shock, etc.)], renal diseases (e.g. diabetes mellitus, diabetic nephropathy, ischemic acute renal failure, etc.) organ disorders associated with ischemia or ischemic reperfusion [(e.g. heart muscle ischemic reperfusion associated disorders, acute renal failure, or disorders induced by surgical treatment such as coronary artery bypass grafting (CABG) surgeries, vascular surgeries, organ transplantation, non-cardiac surgeries or percutaneous transluminal coronary angioplasty (PTCA)], cerebrovascular diseases (e.g., ischemic stroke, hemorrhagic stroke, etc.), cerebro ischemic disorders (e.g., disorders associated with cerebral infarction, disorders caused after cerebral apoplexy as sequelae, or cerebral edema.

The compounds of this invention can also be used as an agent for myocardial protection during coronary artery bypass grafting (CABG) surgeries, vascular surgeries, percutaneous transluminal coronary angioplasty (PTCA), PTCI, organ transplantation, or non-cardiac surgeries.

Preferably, the A₃ receptor agonists can be used as agents for myocardial protection before, during, or after coronary artery bypass grafting (CABG) surgeries, vascular surgeries, percutaneous transluminal coronary angioplasty (PTCA), organ transplantation, or non-cardiac surgeries.

Preferably, the A₃ receptor agonists can be used as agents for myocardial protection in patients presenting with ongoing cardiac (acute coronary syndromes, e.g. myocardial infarction or unstable angina) or cerebral ischemic events (e.g., stroke).

Preferably, the A₃ receptor agonists can be used as agents for chronic myocardial protection in patients with diagnosed coronary heart disease (e.g. previous myocardial infarction or unstable angina) or patients who are at high risk for myocardial infarction (e.g., age greater than 65 and two or more risk factors for coronary heart disease). Accordingly, the A₃ receptor agonists reduce mortality.

6.1.1 Combination Therapy

The invention encompasses combination therapies by administration of one or more compounds of the invention in combination with administration of one or more other therapies that are traditionally used for the treatment and/or prevention of the particular disease or disorder being treated or prevented.

Prophylactic and therapeutic compounds that may be used in the methods and compositions of the invention include, but are not limited to, proteinaceous molecules, including, but not limited to, peptides, polypeptides, proteins, including post-translationally modified proteins, antibodies, etc.; small molecules (less than 1000 daltons), inorganic or organic compounds; nucleic acid molecules including, but not limited to, double-stranded or single-stranded DNA, double-stranded or single-stranded RNA, as well as triple helix nucleic acid molecules. Prophylactic and therapeutic compounds can be derived from any known organism (including, but not limited to, animals, plants, bacteria, fungi, and protista, or viruses) or from a library of synthetic molecules. In certain embodiments, one or more compounds of the invention are administered to a mammal, preferably a human, concurrently with one or more other therapeutic agents useful for the treatment of a disorder. The term “concurrently” is not limited to the administration of prophylactic or therapeutic agents at exactly the same time, but rather it is meant that compounds of the invention and the other agent are administered to a subject in a sequence and within a time interval such that the compounds of the invention can act together with the other agent to provide an increased benefit than if they were administered otherwise. For example, each prophylactic or therapeutic agent may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route.

In various embodiments, the prophylactic or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more components are administered within the same patient visit.

The dosage amounts and frequencies of administration provided herein are encompassed by the terms therapeutically effective and prophylactically effective. The dosage and frequency further will typically vary according to factors specific for each patient depending on the specific therapeutic or prophylactic agents administered, the severity and type of disease, the route of administration, as well as age, body weight, response, and the past medical history of the patient. Suitable regimens can be selected by one skilled in the art by considering such factors and by following, for example, dosages reported in the literature and recommended in the Physician's Desk Reference (58^{th} ed., 2004).

The invention encompasses combination therapies with all currently known (or any method to be developed in the future) methods for treatment of ischemic conditions such as those disclosed in U.S. Pat. Nos. 6,294,579 B1; 6,436,654; 6,22,018; 6,562,799; 5,985,947; 6,544,950; Lazarus et al., “Environmental Health Perspectives”, Vol. 102, No. 4, pages 648-654 (1994); all of which are incorporated herein by reference in their entireties. Current treatments for ischemia encompass behavioral changes, drug therapy, and/or surgical intervention.

The invention encompassess use of other cardiovascular agents and salts thereof (e.g., agents having a cardiovascular effect) in the methods and compositions of the invention when the disease or condition is related to ischemia or hypoxia of cardiac tissue, including but not limited tobeta.-blockers (e.g., acebutolol, atenolol, bopindolol, labetolol, mepindolol, nadolol, oxprenol, pindolol, propranolol, sotalol), calcium channel blockers (e.g., amlodipine, nifedipine, nisoldipine, nitrendipine, verapamil), potassium channel openers, adenosine, adenosine agonists, sodium-hydrogen exchanger type 1 (NHE-1) inhibitors, ACE inhibitors (e.g., captopril, enalapril), nitrates (e.g., isosorbide dinitrate, isosorbide 5-mononitrate, glyceryl trinitrate), diuretics (e.g., hydrochlorothiazide, indapamide, piretanide, xipamide), glycosides (e.g., digoxin, metildigoxin), thrombolytics (e.g. tPA), platelet inhibitors (e.g., reopro), aspirin, dipyridamol, potassium chloride, clonidine, prazosin, pyruvate dehydrogenase kinase inhibitors (e.g., dichloroacetate), pyruvate dehydrogenase complex activators, biguanides (e.g., metformin) or other adenosine A₃ receptor agonists. Other cardiovascular agents include angiotensin II (All) receptor antagonists, C5a inhibitors, soluble complement receptor type 1 (sCR1) or analogues, partial fatty acid oxidation (PFOX) inhibitors (specifically, ranolazine), acetyl CoA carboxylase activators, malonyl CoA decarboxylase inhibitors, 5′AMP-activated protein kinase (AMPK) inhibitors, adenosine nucleoside inhibitors, anti-apoptotic agents (e.g., caspase inhibitors), monophosphoryl lipid A or analogues, nitric oxide synthase activators/inhibitors, protein kinase C activators (specifically, protein kinase E), protein kinase delta inhibitor, poly (ADP ribose) synthetase (PARS, PARP) inhibitors, metformin (gluconeogenesis inhibitors, insulin sensitizers), endothelin converting enzyme (ECE) inhibitors, endothelin ETA receptor antagonists, (thrombin activated fibrinolytic inhibitor) TAFI inhibitors and Na/Ca exchanger modulators.

The compositions and methods of the invention can optionally include other therapeutically active ingredients, such as antibiotics, antivirals, healing promotion agents, anti-inflammatory agents, immunosuppressants, growth factors, anti-metabolites, cell adhesion molecules (CAMs), antibodies, vascularizing agents, and anesthetics/analgesics, anticoagulants, such as an RGD peptide-containing compound, heparin, rapamycin, antithrombin compounds, platelet receptor antagonists, an anti-thrombin antibody, an anti-platelet receptor antibody, aspirin, a prostaglandin inhibitor, a platelet inhibitor, antisense DNA, antisense RNA, a cholesterol-lowering agent, a vasodilating agent, or an agent that interferes with an endogenous vasoactive mechanism. Other examples of other active agents include an anti-inflammatory agent, an anti-platelet or fibrinolytic agent, an anti-neoplastic agent, an anti-allergic agent, an anti-rejection agent, an anti-microbial or anti-bacterial or anti-viral agent, a hormone, a vasoactive substance, an anti-invasive factor, a lymphokine, a radioactive agent or gene therapy drug.

6.2 Compositions and Methods of Administering

The invention provides methods and pharmaceutical compositions comprising compounds of the invention. The invention also provides methods of treatment, prophylaxis, and amelioration of one or more symptoms associated with a disease, disorder or disease by administering to a subject an effective amount of compound of the invention. In a specific embodiment, the subject is an animal, preferably a mammal such as non-primate (e.g., cows, pigs, horses, cats, dogs, rats etc.) and a primate (e.g., monkey such as, a cynomolgous monkey and a human). In a preferred embodiment, the subject is a human.

The compositions of the invention include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, compositions of the invention comprise a prophylactically or therapeutically effective amount of compounds of the invention and a pharmaceutically acceptable carrier.

In one particular embodiment, the pharmaceutical composition comprises of a therapeutically or prophylactically effective amount of an A3 receptor agonist and a pharmaceutically acceptable carrier. In another particular embodiment, the pharmaceutical composition comprises of a therapeutically or prophylactically effective amount of an A₃ receptor agonist and a pharmaceutically acceptable carrier, optionally further comprising one or more additional therapeutic or prophylactic agents.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include, but are not limited to those formed with anions such as those derived from hydrochloric, phosphoric, acetic, oxalic, tartaric acids, etc., and those formed with captions such as those derived from sodium, potassium, ammonium, calcium, ferric hydroxides, isopropylamine, triethylamine, 2-ethylamino ethanol, histidine, procaine, etc.

The compounds described above are preferably administered in formulations including an active compound, i.e., an adenosine A₃ receptor agonist, together with an acceptable carrier for the mode of administration. Suitable pharmaceutically acceptable carriers are known to those of skill in the art. The compositions can optionally include other therapeutically active agents. Other optional ingredients include antivirals, antibacterials, anti-inflammatories, analgesics, and immunosuppresants. The carrier must be pharmaceutically acceptable in the sense of being compatible with the other ingredients of the formulation and not deleterious to the recipient thereof.

The compounds of the inventione, e.g., high affinity adenosine A₃ receptor agonist can be administered in a physiologically acceptable diluent in a pharmaceutical carrier, such as a sterile liquid or mixture of liquids, including water, saline, aqueous dextrose and related sugar solutions, an alcohol (e.g., ethanol, isopropanol, or hexadecyl alcohol), glycols (e.g., propylene glycol or polyethylene glycol), glycerol ketals, (e.g., 2,2-dimethyl 1,3-dioxolane-4-methanol), ethers, (e.g., poly(ethyleneglycol) 400), an oil, a fatty acid, a fatty acid ester or glyceride, or an acetylated fatty acid glyceride with or without the addition of a pharmaceutically acceptable surfactant (e.g., a soap or a detergent), a suspending agent, such as pectin, carbomers, methylcellulose, hydroxypropylmethylcellulose, or carboxymethylcellulose, or emulsifying agents and other pharmaceutical excipients and adjuvants.

The formulations can include carriers suitable for oral, rectal, topical or parenteral (including subcutaneous, intramuscular and intravenous) administration. Preferred carriers are those suitable for oral or parenteral administration.

Formulations suitable for parenteral administration conveniently include sterile aqueous preparation of the active compound which is preferably isotonic with the blood of the recipient. Thus, such formulations may conveniently contain distilled water, 5% dextrose in distilled water or saline. Useful formulations also include concentrated solutions or solids containing the compounds which upon dilution with an appropriate solvent give a solution suitable for parental administration above.

Formulations suitable for parenteral administration include but are not limited to aqueous and non-aqueous solutions, isotonic sterile injection solutions, which may comprise anti-oxidants, buffers, bacteriostats, and solutes that render the formulation isotonic with the blood of the intended recipient, and aqueous and non-aqueous sterile suspensions that may comprise suspending agents, solubilizers, thickening agents, stabilizers, and preservatives.

The parenteral formulations will typically contain from about 0.5 to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The compounds of the inventione, e.g., high affinity adenosine A₃ receptor agonists may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986); which are incorporated herein by reference in their entireties.

For enteral administration, the compound can be incorporated into an inert carrier in discrete units such as capsules, cachets, tablets or lozenges, each containing a predetermined amount of the active compound; as a powder or granules; or a suspension or solution in an aqueous liquid or non-aqueous liquid, e.g., a syrup, an elixir, an emulsion or a draught. Suitable carriers may be starches or sugars and include lubricants, flavorings, binders, and other materials of the same nature.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch.

Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

A tablet may be made by compression or molding, optionally with one or more accessory ingredients. Compressed tablets may be prepared by compressing in a suitable in a machine the active compound in a free-flowing form, e.g., a powder or granules, optionally mixed with accessory ingredients, e.g., binders, lubricants, inert diluents, surface active or dispersing agents. Molded tablets may be made by molding in a suitable machine, a mixture of the powdered active compound with any suitable carrier.

A syrup or suspension may be made by adding the active compound to a concentrated, aqueous solution of a sugar, e.g., sucrose, to which may also be added any accessory ingredients. Such accessory ingredients) may include flavoring, an agent to retard crystallization of the sugar or an agent to increase the solubility of any other ingredient, e.g., as a polyhydric alcohol, for example, glycerol or sorbitol.

The compounds can also be administered locally by topical application of a solution, ointment, cream, gel, lotion or polymeric material (for example, a Pluronic™, BASF), which may be prepared by conventional methods known in the art of pharmacy. In addition to the solution, ointment, cream, gel, lotion or polymeric base and the active ingredient, such topical formulations may also contain preservatives, perfumes, and additional active pharmaceutical agents. Topical formulations for high affinity adenosine A₃ receptor agonists include ointments, creams, gels and lotions that may be prepared by conventional methods known in the art of pharmacy. Such topical formulation may also furhter comprise preservatives, perfumes, and additional active pharmaceutical agents.

Oils, which can be used in parenteral formulations include petroleum, animal, vegetable, and synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-beta-aminopropionates, and 2-alkyl-imidazoline quaternary ammonium salts, and (e) mixtures thereof.

Additionally, the compounds of the invention, e.g., high affinity adenosine A₃ receptor agonist may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Formulations for rectal administration may be presented as a suppository with a conventional carrier, e.g., cocoa butter or Witepsol S55 (trademark of Dynamite Nobel Chemical, Germany), for a suppository base.

The formulations may conveniently be presented in unit dosage form and may be prepared by any of the methods well known in the art of pharmacy. All methods include the step of bringing the active compound into association with a carrier which constitutes one or more accessory ingredients. In general, the formulations are prepared by uniformly and intimately bringing the active compound into association with a liquid carrier or a finely divided solid carrier and then, if necessary, shaping the product into desired unit dosage form.

In addition to the aforementioned ingredients, the formulations of this invention may further include one or more cytotoxic agent as well as one or more optional accessory ingredient(s) utilized in the art of pharmaceutical formulations, e.g., diluents, buffers, flavoring agents, binders, surface active agents, thickeners, lubricants, suspending agents, preservatives (including antioxidants) and the like.

Various delivery systems are known and can be used to administer a composition comprising compounds of the invention, e.g., encapsulation in liposomes, microparticles, microcapsules.

In some embodiments, the compounds of the invention are formulated in liposomes for targeted delivery of the compounds of the invention. Liposomes are vesicles comprised of concentrically ordered phopsholipid bilayers which encapsulate an aqueous phase. Liposomes typically comprise various types of lipids, phospholipids, and/or surfactants. The components of liposomes are arranged in a bilayer configuration, similar to the lipid arrangement of biological membranes. Liposomes are particularly preferred delivery vehicles due, in part, to their biocompatibility, low immunogenicity, and low toxicity. Methods for preparation of liposomes are known in the art and are encompassed within the invention, see, e.g., Epstein et al., 1985, Proc. Natl. Acad. Sci. USA, 82: 3688; Hwang et al., 1980 Proc. Natl. Acad. Sci. USA, 77: 4030-4; U.S. Pat. Nos. 4,485,045 and 4,544,545; all of which are incorporated herein by reference in their entirety. The invention also encompasses methods of preparing liposomes with a prolonged serum half-life, i.e., enhanced circulation time, such as those disclosed in U.S. Pat. No. 5,013,556. Preferred liposomes used in the methods of the invention are not rapidly cleared from circulation, i.e., are not taken up into the mononuclear phagocyte system (MPS). The invention encompasses sterically stabilized liposomes which are prepared using common methods known to one skilled in the art. Although not intending to be bound by a particular mechanism of action, sterically stabilized liposomes contain lipid components with bulky and highly flexible hydrophilic moieties, which reduces the unwanted reaction of liposomes with serum proteins, reduces oposonization with serum components and reduces recognition by MPS. Sterically stabilized liposomes are preferably prepared using polyethylene glycol. For preparation of liposomes and sterically stabilized liposome see, e.g., Bendas et al., 2001 BioDrugs, 15(4): 215-224; Allen et al., 1987 FEBS Lett. 223: 42-6; Klibanov et al., 1990 FEBS Lett., 268: 235-7; Blum et al., 1990, Biochim. Biophys. Acta., 1029: 91-7; Torchilin .et al., 1996, J. Liposome Res. 6: 99-116; Litzinger et al., 1994, Biochim. Biophys. Acta, 1190: 99-107; Maruyama et al., 1991, Chem. Pharm. Bull., 39: 1620-2; Klibanov et al., 1991, Biochim Biophys Acta, 1062; 142-8; Allen et al., 1994, Adv. Drug Deliv. Rev, 13: 285-309; all of which are incorporated herein by reference in their entirety. The invention also encompasses liposomes that are adapted for specific organ targeting, see, e.g., U.S. Pat. No. 4,544,545. Particularly useful liposomes for use in the compositions and methods of the invention can be generated by reverse phase evaporation method with a lipid composition comprising phosphatidylcholine, cholesterol, and PEG derivatized phosphatidylethanolamine (PEG-PE). Liposomes are extruded through filters of defined pore size to yield liposomes with the desired diameter. In some embodiments, a fragment of an antibody of the invention, e.g., F(ab′), may be conjugated to the liposomes using previously described methods, see, e.g., Martin et al., 1982, J. Biol. Chem. 257: 286-288, which is incorporated herein by reference in its entirety.

Methods for preparing liposomes and microspheres for administration to a patient are well known to those of skill in the art. U.S. Pat. No. 4,789,734, the contents of which are hereby incorporated by reference, describes methods for encapsulating biological materials in liposomes. essentially, the material is dissolved in an aqueous solution, the appropriate phospholipids and lipids added, along with surfactants if required, and the material dialyzed or sonicated, as necessary. A review of known methods is provided by G. Gregoriadis, Chapter 14, “Liposomes,” Drug Carriers in Biology and Medicine, pp. 287-341 (Academic Press, 1979), which is incorporated herein by reference in its entirety.

Microspheres formed of polymers or proteins are well known to those skilled in the art, and can be tailored for passage through the gastrointestinal tract directly into the blood stream. Alternatively, the compound can be incorporated and the microspheres, or composite of microspheres, implanted for slow release over a period of time ranging from days to months. See, for example, U.S. Pat. Nos. 4,906,474, 4,925,673 and 3,625,214, the contents of which are hereby incorporated by reference.

Preferred microparticles are those prepared from biodegradable polymers, such as polyglycolide, polylactide and copolymers thereof. Those of skill in the art can readily determine an appropriate carrier system depending on various factors, including the desired rate of drug release and the desired dosage.

In another embodiment, the compositions can be delivered in a vesicle, in particular a liposome (See Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 3 17-327; see generally ibid.).

In yet another embodiment, the compositions can be delivered in a controlled release or sustained release system. Any technique known to one of skill in the art can be used to produce sustained release formulations comprising one or more compounds of the invention. See, e.g., U.S. Pat. No. 4,526,938; PCT publication WO 91/05548; PCT publication WO 96/20698; Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397; Cleek et al., 1997, “Biodegradable Polymeric Carriers for a bFGF Antibody for Cardiovascular Application,” Pro. Int'l. Symp. Control. Rel. Bioact. Mater. 24:853-854; and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in its entirety. In one embodiment, a pump may be used in a controlled release system (See Langer, supra; Sefton, 1987, CRC Crit. Ref. Biomed Eng. 14:20; Buchwald et al., 1980, Surgery 88:507; and Saudek et al., 1989, N. Engl. J. Med 321:574). In another embodiment, polymeric materials can be used to achieve controlled release of compounds (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; See also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In yet another embodiment, a controlled release system can be placed in proximity of the therapeutic target (e.g., the lungs), thus requiring only a fraction of the systemic dose (see, e.g., Goodson, in Medical Applications of Controlled Release, supra, vol. 2, pp. 115-138 (1984)). In another embodiment, polymeric compositions useful as controlled release implants are used according to Dunn et al. (See U.S. Pat. No. 5,945,155). This particular method is based upon the therapeutic effect of the in situ controlled release of the bioactive material from the polymer system. The implantation can generally occur anywhere within the body of the patient in need of therapeutic treatment. In another embodiment, a non-polymeric sustained delivery system is used, whereby a non-polymeric implant in the body of the subject is used as a drug delivery system. Upon implantation in the body, the organic solvent of the implant will dissipate, disperse, or leach from the composition into surrounding tissue fluid, and the non-polymeric material will gradually coagulate or precipitate to form a solid, microporous matrix (See U.S. Pat. No. 5,888,533). Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938; International Publication Nos. WO 91/05548 and WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. Rel. Bioact. Mater. 24:853-854; and Lam et al., 1997, Proc. Int'l. Symp. Control Rel. Bioact. Mater. 24:759-760, each of which is incorporated herein by reference in its entirety.

Methods of administering a compound of the invention include, but are not limited to, parenteral administration (e.g., intradermal, intramuscular, intraperitoneal, intravenous and subcutaneous), epidural, and mucosal (e.g., intranasal and oral routes). In a specific embodiment, the compounds of the invention are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968; 5,985, 20; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903, each of which is incorporated herein by reference in its entirety.

In one embodiment, the compounds are administered intravenously in a liposome or microparticle with a size such that the particle can be delivered intraveneously, but gets trapped in a capillary bed around a growing tumor. Suitable particle sizes for this embodiment are those currently used, for example, in liposomes sold under the name DaunoXome™, which are believed to be between about 200 and 500 μm. The compounds are then released locally, over time, at the location of the tumor.

In another embodiment, the compounds are administered in a tissue coating, preferably a polymeric tissue coating, more preferably, a biodegradable tissue coating, which is applied to the site at which a tumor is surgically removed. Suitable polymeric materials are disclosed, for example, in U.S. Pat. No. 5,410,016 to Hubbell et al., the contents of which are hereby incorporated by reference.

The polymeric barrier, in combination with the adenosine A₃ agonists, and optionally in combination with other angiogenic agents.

The amount of the composition of the invention which will be effective in the treatment, prevention or amelioration of one or more symptoms associated with a disorder can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems.

In a specific embodiment, it may be desirable to administer the pharmaceutical compositions of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

Treatment of a subject with a therapeutically or prophylactically effective amount of compounds of the invention can include a single treatment or, preferably, can include a series of treatments. In a preferred example, a subject is treated with compounds of the invention in the range of between about about 0.1 μg/kg to about 100 mg/kg, about 0.1 μg/kg to about 500 mg/kg, about 0.1 μg/kg to about 1 g/kg, about 100 ug/kg to about 500 mg/kg, about 100 ug/kg to about 1 g/kg, about 1 mg/kg to about 100 mg/kg, about 1 mg/kg to about 500 mg/kg, about 1 mg/kg to about 1 g/kg of the patient's body weight, one time per week for between about 1 to 10 weeks, preferably between 2 to 8 weeks, more preferably between about 3 to 7 weeks, and even more preferably for about 4, 5, or 6 weeks. In other embodiments, the pharmaceutical compositions of the invention are administered once a day, twice a day, or three times a day. In other embodiments, the pharmaceutical compositions are administered once a week, twice a week, once every two weeks, once a month, once every six weeks, once every two months, twice a year or once per year. It will also be appreciated that the effective dosage of the compounds used for treatment may increase or decrease over the course of a particular treatment. The amount of a compound required to be effective as an agonist of adenosine A₃ receptors will, of course, vary with the active moiety selected, the individual mammal being treated and is ultimately at the discretion of the medical or veterinary practitioner. The factors to be considered include the binding affinity of the active, the route of administration, the nature of the formulation, the mammal's body weight, surface area, age and general condition, and the particular compound to be administered.

The total daily dose may be given as a single dose, multiple doses, e.g., two to six times per day, or by intravenous infusion for a selected duration. Dosages above or below the range cited above are within the scope of the present invention and may be administered to the individual patient if desired and necessary.

The invention also provides that the compounds of the invention are packaged in a hermetically sealed container such as an ampoule or sachette indicating the quantity of antibody. In one embodiment, the compounds of the invention are supplied as a dry sterilized lyophilized powder or water free concentrate in a hermetically sealed container and can be reconstituted, e.g., with water or saline to the appropriate concentration for administration to a subject. Preferably, the compounds of the invention are supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, or at least 75 mg. The lyophilized compounds of the invention should be stored at between 2 and 8° C. in their original container and the compounds should be administered within 12 hours, preferably within 6 hours, within 5 hours, within 3 hours, or within 1 hour after being reconstituted. In an alternative embodiment, compounds of the invention are supplied in liquid form in a hermetically sealed container indicating the quantity and concentration of the compound. Preferably, the liquid form of the compounds are supplied in a hermetically sealed container at least 1 mg/ml, more preferably at least 2.5 mg/ml, at least 5 mg/ml, at least 8 mg/ml, at least 10 mg/ml, at least 15 mg/kg, at least 25 mg/ml, at least 50 mg/ml, at least 100 mg/ml, at least 150 mg/ml, at least 200 mg/ml of the compounds.

The formulations may conveniently be presented in unit dosage form and may be prepared by any of the methods well known in the art of pharmacy. All methods include the step of bringing the active compound into association with a carrier which constitutes one or more accessory ingredients. In general, the formulations are prepared by uniformly and intimately bringing the active compound into association with a liquid carrier or a finely divided solid carrier and then, if necessary, shaping the product into desired unit dosage form.

In addition to the aforementioned ingredients, the formulations may further include one or more optional accessory ingredient(s) utilized in the art of pharmaceutical formulations, e.g., diluents, buffers, flavoring agents, binders, surface active agents, thickeners, lubricants, suspending agents, preservatives (including antioxidants) and the like.

The formulations include, but are not limited to, those suitable for oral, rectal, topical or parenteral (including subcutaneous, intramuscular and intravenous) administration. Preferred are those suitable for oral or parenteral administration.

The pharmaceutically acceptable carriers described herein, for example, vehicles, adjuvants, excipients, or diluents, are well known to those who are skilled in the art and are readily available to the public. It is preferred that the pharmaceutically acceptable carrier be one which is chemically inert to the active compounds and one which has no detrimental side effects or toxicity under the conditions of use.

The choice of carrier will be determined in part by the particular active agent, as well as by the particular method used to administer the composition. Accordingly, there are a wide variety of suitable formulations of the pharmaceutical composition of the present invention. The following formulations for oral, aerosol, parenteral, subcutaneous, intravenous, intraarterial, intramuscular, interperitoneal, intrathecal, rectal, and vaginal administration are merely exemplary and are in no way limiting.

The compounds of the invention, e.g., high affinity adenosine A₃ receptor antagonist, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

6.3 Characterization and Demonstration of Therapeutic/Prophylactic Utility

Several aspects of the pharmaceutical compositions, prophylactic or therapeutic agents of the invention are preferably tested in vitro, e.g., in a cell culture system, and then in vivo, e.g., in an animal model organism, such as a rodent animal model system, for the desired therapeutic activity prior to use in humans. Combinations of prophylactic and/or therapeutic agents can be tested in suitable animal model systems prior to use in humans. Such animal model systems include, but are not limited to, rats, mice, chicken, cows, monkeys, pigs, dogs, rabbits, etc. Any animal system well-known in the art may be used. In a specific embodiment of the invention, combinations of prophylactic and/or therapeutic agents are tested in a mouse model system. Such model systems are widely used and well-known to the skilled artisan. Prophylactic and/or therapeutic agents can be administered repeatedly. Several aspects of the procedure may vary such as the temporal regime of administering the prophylactic and/or therapeutic agents, and whether such agents are administered separately or as an admixture.

Once the prophylactic and/or therapeutic agents of the invention have been tested in an animal model they can be tested in clinical trials to establish their efficacy. Establishing clinical trials will be done in accordance with common methodologies known to one skilled in the art, and the optimal dosages and routes of administration as well as toxicity profiles of the compositions of the invention can be established using routine experimentation.

Toxicity and efficacy of the prophylactic and/or therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The anti-ischemic activity of the therapies used in accordance with the present invention also can be determined by using various experimental animal models for the study of ischemia. Animal models have been established that mimic the symptoms of both global and focal cerebral ischemia, most notably, the gerbil model of global ischemia produced by transient occlusion of carotid arteries of the neck (see, e.g., Kirino et al., 1982, Brain Res. 239:57-69), the rat four-vessel occlusion model for ischemia (Pulsinelli et al., 1979, Stroke 10:267-272), the MCAO microfilament of focal ischemia (Tamura et al., 1981, Journal Cereb. Blood Flow Metab. 1:53); all of which are incorporated herein by reference in their entireties.

The protocols and compositions of the invention are preferably tested in vitro, and then in vivo, for the desired therapeutic or prophylactic activity, prior to use in humans. Therapeutic agents and methods may be screened using cells of a tumor or malignant cell line. Compounds for use in therapy can be tested in suitable animal model systems prior to testing in humans, including but not limited to in rats, mice, chicken, cows, monkeys, rabbits, hamsters, etc., for example, the animal models described above. The compounds can then be used in the appropriate clinical trials.

The therapeutic utility of the A₃ receptor agonist for myocardial protection during surgery or mycardial protection in patients with ongoing cardiac or cerebral ischemic events or chronic cardioprotection in patients with diagnosed coronary heart disease, or at risk for coronary heart disease, cardiac dysfunction may be determined using standard assays known to the skilled artisan, such as conventional preclinical cardioprotection assays, see, e.g., the in vivo assay in Klein et al., 1995, Circulation 92:912-917; the isolated heart assay in Scholz et al., 1995, Cardiovascular Research 29:260-268; the antiarrhythmic assay in Yasutake et al., 1994, Am. J. Physiol., 36:H2430-H2440; the NMR assay in Kolke et al., 1996, J. Thorac. Cardiovasc. Surg. 112: 765-775; which are incorporated herein by reference in their entireties. Such assays also provide a means whereby the activities of the compounds of this invention can be compared with the activities of other known compounds. The results of these comparisons are useful for determining dosage levels in mammals, including humans, for the treatment of such diseases.

The therapeutic effects of the A₃ receptor agonist in preventing heart tissue damage resulting from an ischemic insult can be demonstrated in an in vitro assay such as those disclosed by Liu et al. (Cardiovasc. Res., 28:1057-1061, 1994; which is incorporated herein by reference in its entirety). Cardioprotection, as indicated by a reduction in infarcted myocardium, can be induced pharmacologically using adenosine receptor agonists in isolated, retrogradely perfused rabbit hearts as an in vitro model of myocardial ischemic preconditioning. The therapeutic effects of the A₃ receptor agonist in preventing heart tissue damage otherwise resulting from an ischemic insult can also be demonstrated in vivo using methods presented in Liu et al. (Circulation, Vol. 84:350-356, 1991, which is incorporated herein by reference in its entirety). The in vivo assay tests the cardioprotection of the test compound relative to the control group which receives saline vehicle. Cardioprotection, as indicated by a reduction in infarcted myocardium, can be induced pharmacologically using intravenously administered adenosine receptor agonists in intact, anesthetized rabbits studied as an in situ model of myocardial ischemic preconditioning (Liu et al., Circulation 84:350-356, 1991; which is incorporated herein by reference in its entirety). The in vivo assay tests whether compounds can pharmacologically induce cardioprotection, i.e., reduced myocardial infarct size, when parenterally administered to intact, anesthetized rabbits. The effects of the compounds of this invention can be compared to ischemic preconditioning using the A₃ adenosine agonist, N⁶-1-(phenyl-2R-isopropyl) adenosine (PIA) that has been shown to pharmacologically induce cardioprotection in intact anesthetized rabbits studied in situ.

The A₃ receptor agonists can be tested for their utility in reducing or preventing ischemic injury in non-cardiac tissues, for example, the brain, or the liver, utilizing procedures reported in the scientific literature. The A₃ receptor agonist in such tests can be administered by the preferred route and vehicle of administration and at the preferred time of administration either prior to the ischemic episode, during the ischemic episode, following the ischemic episode (reperfusion period). The benefit of the invention to reduce ischemic brain damage can be demonstrated, for example, in mammals using the method of Park, et al (Ann. Neurol. 1988;24:543-55 l; Nakayama et al. Neurology 1988,38:1667-1673; Memezawa et al. Stroke 1992,23:552-559; Folbergrova et al., Proc. Natl. Acad. Sci 1995,92:5057-5059; and Gotti et al. Brain Res. 1990,522:290-307; which are incorporated herein by reference in their entireties).

6.3.1 Adenosine Receptor Based Assays

The activity and selectivity of the compounds of the invention as adenosine A3 agonists can be readily determined using no more than routine experimentation using any of the assays disclosed herein or known to one skilled in the art. Since the A₁ and A_{2A} receptors express similar pharmacology between humans and rodents, endogenous receptors from the rat can be used for the A₁ and A_{2A} binding assays.

An exemplary rat A₁ and A_{2A} Adenosine receptor binding assay may comprise the following steps. Membrane preparations: Male Wistar rats (200-250 g) can be decapitated and the whole brain (minug brainstem, striatum and cerebellum) dissected on ice. The brain tissues can be disrupted in a Polytron (setting 5) in 20 vols of 50 mM Tris HCl, pH 7.4. The homogenate can then be centrifuged at 48,000 g for 10 min and the pellet resuspended in Tris-HCL containing 2 IU/ml adenosine deaminase, type VI (Sigma Chemical Company, St. Louis, Mo., USA). After 30 min incubation at 37° C., the membranes can be centrifuged and the pellets stored at −70° C. Striatal tissues can be homogenized with a Polytron in 25 vol of 50 mM Tris HCl buffer containing 10 mM MgCl₂ pH 7.4. The homogenate can then be centrifuged at 48,000 g for 10 min at 4° C. and resuspended in Tris HCl buffer containing 2 IU/ml adenosine deaminase. After 30 min incubation at 37° C., membranes can be centrifuged and the pellet stored at −70° C. The radioligand binding assays may comprise the following: Binding of [³H]-DPCPX (1,3-dipropyl-8-cyclopentylxanthine) to rat brain membranes can be performed essentially according to the method previously described by Bruns et al., 1980, Proc. Natl. Acad. Sci. 77, 5547-5551, which is incorporated herein by reference in its entirety. Displacement experiments can be performed in 0.25 ml of buffer containing 1 nM [³H]-DPCPX, 100 ul of diluted membranes of rat brain (100 μg of protein/assay) and at least 6-8 different concentrations of examined compounds. Non specific binding can be determined in the presence of 10 uM of CHA (N6 cyclohexyladenosine) and this is always ≦10% of the total binding. Incubation times are typically 120 min at 25° C.

Radioligand binding assays may comprise the following: Binding of [³H]-SCH 58261 (5-amino-7-(2-phenylethyl)-2-(2-furyl)-pyrazolo[4,3-e]-1,2,4-triazolo[1,5-c]pyrimidine) to rat striatal membranes (100 ug of protein/assay) can be performed according to methods described in Zocchi et al., 1996, J. Pharm. and Exper. Ther. 276:398-404, which is incorporated herein by reference in its entirety. In competition studies, at least 6-8 different concentrations of examined compounds should be used. Non specific binding can be determined in the presence of 50 uM of NECA (5′-(N-ethylcarboxamido)adenosine). Incubation time is typically 60 min at 25° C. Bound and free radioactivity can be separated by filtering the assay mixture through Whatman GF/B glass-fiber filters using a Brandel cell harvester (Gaithersburg, Md., USA). The incubation mixture can be diluted with 3 ml of ice-cold incubation buffer, rapidly vacuum filtered and the filter can be washed three times with 3 ml of incubation buffer. The filter bound radioactivity can be measured, for example, by liquid scintillation spectrometry. The protein concentration can be determined, for example, according to a Bio-Rad method (Bradford, 1976, Anal. Biochem. 72:248, which is incorporated herein by reference in its entirety) with bovine albumin as reference standard.

An exemplary assay for human cloned A₃ Adenosine Receptor Binding Assay may comprise the following: Binding assays can be carried out according to methods described in Salvatore et al., 1993, Proc. Natl. Acad. Sci. 90:10365-10369; which is incorporated herein by reference in its entirety. In saturation studies, an aliquot of membranes (8 mg protein/ml) from HEK-293 cells transfected with the human recombinant A3 adenosine receptor (Research Biochemical International, Natick, Mass., USA) can be incubated with 10-12 different concentrations of [¹²⁵I]AB-MECA ranging from 0.1 to 5 nM. Competition experiments can be carried out in duplicate in a final volume of 100 ul in test tubes containing 0.3 nM [¹²⁵I]AB-MECA, 50 mM Tris HCl buffer, 10 mM MgCl₂, pH 7.4 and 20 ul of diluted membranes (12.4 mg protein/ml) and at least 6-8 different concentrations of examined ligands. Incubation time was 60 min at 37° C., according to the results of previous time-course experiments. Bound and free radioactivity were separated by filtering the assay mixture through Whatman GF/B glass-fiber filters using a Brandel cell harvester. Non-specific binding was defined as binding in the presence of 50 uM R-PIA and was about 30% of total binding. The incubation mixture was diluted with 3 ml of ice-cold incubation buffer, rapidly vacuum filtered and the filter was washed three times with 3 ml of incubation buffer. The filter bound radioactivity was counted in a Beckman gamma 5500B gamma counter. The protein concentration can be determined according to a Bio-Rad method with bovine albumin as reference standard.

Data Analysis may be carried out as follows: Inhibitory binding constant, Ki, values can be calculated from those of IC₅₀ according to the Cheng & Prusoff equation (Cheng and Prusoff, 1973, Biochem. Pharmacol. 22:3099-3108), Ki=IC₅₀/(1+[C*]I/K_{D}*), where [C*] is the concentration of the radioligand and K_{D}*its dissociation constant. A weighted non linear least-squares curve fitting program LIGAND (Munson and Rodbard, 1990, Anal. Biochem. 107:220-239) can be used for computer analysis of saturation and inhibition experiments. Data are typically expressed as geometric mean, with 95% or 99% confidence limits in parentheses.

6.3.2 Assays for Measuring Ischemia

The invention encompasses in vitro and in vivo based assays useful for determining the efficacy of the compounds of the invention in the treatment or prevention ischemia-related cellular damage. Any method known in the art for the measuring ischemia-related cellular damage is encompassed within the invention. Any method known in the art for measuring ischemic cell damage, necrotic cell death, apoptotic cell death, may be used in accordance with the methods of the invention.

Once the efficacy of the agonists of the invention is evaluated in an in vitro assay, they can be further validated in in vivo models of ischemia. This section describes exemplary models for this purpose. Persons skilled in the art will appreciate that other models can be substituted for the models described below.

Various in vivo models have been described that produce neuronal ischemia in the central nervous system. Exemplary models include gerbil 2-vessel occlusion model of global ischemia produced by transient occlusion of carotid arteries (Kirino, 1982, Brain Res. 239:57-69), the rat four-vessel occlusion model of global ischemia (Pulsinelli, et al., 1979, Stroke 10:267-272), and the rat middle cerebral artery occlusion (MCAO) model of focal ischemia (Tamura et al., 1981, J Cereb. Blood Flow Metab. 1:53). All of the references cited supra are incorporated herein by reference in their entirety.

Mongolian gerbils have been used as a model for cerebral ischemia and infarction (Kirino, 1982, Brain Res. 239:57-69). The gerbil lacks an interconnection between the carotid and vertebro-basilar circulation such that one can easily produce cerebral ischemia by occlusion of the common carotid arteries of the neck. The gerbil brain subjected to transient bilateral carotid occlusion for no longer than 5 minutes can produce a typical ischemic lesion in the CA1 region of the hippocampus. For clinical comparisons, the ischemia produced in this model has been likened to that produced by cardiac arrest, since all blood flow to the brain is stopped for a fixed period, typically 5-10 minutes.

Although some differences in particular sequelae have been noted between species, gerbils exhibit the same kind of selective regional damage resulting from ischemia as is found in other mammals, including humans. In particular, the characteristic secondary damage observed in the hippocampal CA1 region is similar to that seen in other mammals, including humans. Neurons in this area, and especially pyramidal neurons, exhibit a delayed neuronal death over a period of up to 4 days after ischemic injury.

The rat model encompasses a procedure for producing temporary occlusion and produces an ischemia that mimics conditions in the human brain following cardiac arrest, including a temporary ischemic event, typically 5-30 minutes, which occurs in an unanesthetized state. In most rats, the ischemic event is not accompanied by generalized seizures, and animals that have seizures can be excluded from the study. The occlusion procedure allows the animals to be easily monitored, maintained and analyzed Pulsinelli, et al., 1979, Stroke 10:267-272).

The selective N-type calcium channel blocker, SNX-111, has been demonstrated to be neuroprotective in both the rat 4 vessel occlusion model of ischemia and a model of transient middle cerebral artery occlusion focal ischemia (Buchan, et al., 1994 J Cereb. Blood Flow Metab. 14(6):903-910.)

Animal stroke models with focal cerebral infarction, which have been established in cat, dog, primates, gerbils and rats are believed to be directly relevant to clinical experience. A commonly used focal ischemia model in the rat is the right middle cerebral artery occlusion (MCAO) model developed by Tamura and co-workers. [Hsu et al., 1990, Cerebral Ischemia and Resuscitation 3:47-59, which is incorporated herein by reference in its entirety). Briefly, Male Wistar rats weighing 310-340 g are anaesthetized with 3-3.5% halothane, and orally intubated. Nylon monofilament fishing thread or silicone rubber-coated nylon fishing line, with an outer diameter of approximately 28 mm is used to occlude the middle cerebral artery, by insertion from the external carotid artery, as described in Hsu, et al., 1990. The MCAO model requires no craniectomy and allows easy reperfusion, however, temperature can influence focal ischemic damage due to middle cerebral artery (MCA) occlusion, but this complication can be avoided by anesthesia and/or cooling of awake animals.

Animal models of myocardial infarction are well known in the art. Any of a number of models can be used to validate the efficacy of thecompounds as identified herein. For example, in situ coronary artery occlusion followed by reperfusion in rabbits or dogs is used to assess compounds, where extent of damage to the heart is measured by any of a number of methods, such as magnetic resonance imaging (see, e.g., Kim et al., 1999, Circulation 100(2) 185-192; Pislaru et al., 1999, Circulation 99(5): 690-696; Schwartz, 1999 Am. J. Cardiol. 81(6A): 14D-20D; each of which is incorporated herein by reference in its entirety).

Animal models for COPD and asthma are well known in the art and encompassed herein for assaying the efficacy of the antagonist compounds of the invention. See, e.g., Steiger et al., 1995, J. Am. Respir. Cell Mol. Biol., 12:307-14 and U.S. Pat. No. 6,083,973; Temann et al., 1997, Am. J. Respir. Cell Mol. Biol. 16:471-8; Szelenyi and Marx, 2001, Arzneimittelforschung 51:1004-14; all of which are incorporated herein by reference in their entireties.

6.3.3 Methods For Determining and Measuring HIF-1 Alpha Levels

The invention encompasses methods to assess quantitative and qualitative aspects of HIF-1α expression. Techniques well known in the art, e.g., quantitative or semi-quantitative RT PCR or Northern blot, can be used to measure expression levels of HIF-1α. Methods that describe both qualitative and quantitative aspects of HIF-1α gene or gene product expression are described in detail in the examples infra. The measurement of HIF-1α gene expression levels can include measuring naturally occurring HIF-1α transcripts and variants thereof as well as non-naturally occurring variants thereof, however for the diagnosis and/or prognosis of diseases or disorders in a subject the HIF-1α gene product is preferably a naturally occurring HIF-1α gene product or variant thereof. Thus, the invention relates to methods of measuring the expression of the HIF-1α gene in a subject.

Any method known in the art for detecting and/or quantitating an HIF-1α level may be used in the methods and kits of the invention, a number of which are exemplified herein. Particularly preferred are methods known in the art for detecting and/or quantitating an HIF-1α activity or an HIF-1α related activity, e.g., phosphorylation of downstream effector molecules in the HIF-1α pathway. In some embodiments, the invention encompasses measuring an HIF-1α activity or an HIF-1α related activity including but not limited to, measuring an activity of one or more downstream effectors of an HIF-1α signaling casade. Measuring an HIF-1α activity or an HIF-1α related activity can be done using any of the methods disclosed herein or any standard method known to one skilled in the art.

In other embodiments, the invention encompasses quantitation of a nucleic acid encoding HIF-1α in a sample obtained from a subject using methods disclosed herein or any standard method known in the art.

In yet other embodiments, the invention encompasses quantitation of HIF-1α protein in a sample obtained from a subject with a disease or disorder. Any method known in the art for the detection and quantitation of a HIF-1α protein is encompassed within the present invention.

6.3.3.1 Detection of Nucleic Acid Molecules

The methods and kits of the invention encompass detection and/or quantitation of a nucleic acid sequence encoding HIF-1α in a sample obtained from a subject. In certain embodiments, the invention provides methods for amplifying a specific HIF-1α nucleic acid sequence in a sample obtained from a subject with a disease or disorder and detecting and/or quantitating the same. Nucleic acids encoding HIF-1α are well known in the art. See, for example, Wang et al., 1995, Proc. Natl. Acad. Sci. USA, 92: 5510-4; and WO 96/39426 each of which is incorporated herein by reference in their entireties.

The methods and kits of the invention may use any nucleic acid amplification or detection method known to one skilled in the art, such as those described in U.S. Pat. Nos. 5,525,462; 6,528,632; 6,344,317; 6,114,117; 6,127,120; 6,448,001; all of which are incorporated herein by reference in their entirety.

In some embodiments, the nucleic acid encoding an HIF-1α is amplified by PCR amplification using methodologies known to one skilled in the art. One of skill in the art will recognize, however, that amplification of target sequences (i.e., nucleic acid sequences encoding HIF-1α) in a sample obtained from a subject with a disease or disorder can be accomplished by any known method, such as ligase chain reaction (LCR), QP-replicase amplification, transcription amplification, and self-sustained sequence replication, each of which provides sufficient amplification. The PCR process is well known in the art and is thus not described in detail herein. For a review of PCR methods and protocols, see, e.g., Innis et al., eds., PCR Protocols, A Guide to Methods and Application, Academic Press, Inc., San Diego, Calif. 1990; which is incorporated herein by reference in its entirety). Also see U.S. Pat. No. 4,683,202; which is incorporated herein by reference in its entirety. PCR reagents and protocols are also available from commercial vendors, such as Roche Molecular Systems.

The invention encompasses methods to determine quantitative and/or qualitative levels of expression of HIF-1α. Any technique known in the art for measuring the expression of an HIF-1α is within the scope of the invention, including but not limited, to quantitative and/or semi-quantitative RT PCR and Northern blot analysis.

In some embodiments, the invention encompasses detecting and/or quantitating an HIF-1α nucleic acid using fluorescence in situ hybridization (FISH) in a sample, preferably a tissue sample, obtained from a subject with an ischemic disease or disorder in accordance with the methods of the invention. FISH is a common methodology used in the art, especially in the detection of specific chromosomal aberrations in tumor cells, for example, to aid in diagnosis and tumor staging. As applied in the methods of the invention, it can also be used as a method for detection and/or quantitation of an HIF-1α nucleic acid. For a review of FISH methodology, see, e.g., Weier et al., 2002, Expert Rev. Mol. Diagn. 2(2): 109-119; Trask et al., 1991, Trends Genet. 7(5): 149-154; and Tkachuk et al., 1991, Genet. Anal. Tech. Appl. 8: 676-74; all of which are incorporated herein by reference in their entirety.

The invention encompasses measuring naturally occurring HIF-1α transcripts and variants thereof as well as non-naturally occurring variants thereof. For the prognosis of an ischemic disorder in a subject using the methods of the invention, the HIF-1α transcript is preferably a naturally occurring HIF-1α transcript.

In some embodiments, the invention relates to methods of prognosis of a disease in a subject by measuring the expression of an HIF-1α transcript in a subject. For example, the decreased level of mRNA encoding an HIF-1α, as compared to a standard, would indicate the increased risk of developing an ischemic condition in said subject.

In one embodiment, the invention encompasses isolating RNA from a sample obtained from a subject with an ischemic disorder, and testing the RNA utilizing hybridization or PCR techniques as described above for determining the level of an HIF-1α. In another embodiment, the invention encompasses synthesizing cDNA from the isolated RNA by reverse transcription. All or part of the resulting cDNA is then used as the template for a nucleic acid amplification reaction, such as a PCR or the like. The nucleic acid reagents used as synthesis initiation reagents (e.g., primers) in the reverse transcription and nucleic acid amplification steps of this method are chosen from among the HIF-1α nucleic acid reagents described below. The preferred lengths of such nucleic acid reagents are at least 9-30 nucleotides. For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

In alternative embodiments, standard Northern analysis techniques known to one skilled in the art can be performed on a sample obtained from a subject with a disease or disorder. The preferred length of a probe used in Northern analysis is 9-50 nucleotides. Utilizing such techniques, quantitative as well as size related differences among HIF-1α transcripts can also be detected.

In alternative embodiments, the invention encompasses gene expression assays in situ, i.e., directly upon tissue sections (fixed and/or frozen) of patient tissue obtained from biopsies or resections, such that no nucleic acid purification is necessary. Nucleic acid reagents such as those described below may be used as probes and/or primers for such in situ procedures (see, e.g., Nuovo, G. J., 1992, PCR In Situ Hybridization: Protocols And Applications, Raven Press, NY, which is incorporated herein by reference in its entirety).

The target HIF-1α nucleic acids of the invention can also be detected using other standard techniques well known to those of skill in the art. Although the detection step is typically preceded by an amplification step, amplification is not necessarily required in the methods of the invention. For instance, the HIF-1α nucleic acids can be identified by size fractionation (e.g., gel electrophoresis). The presence of different or additional bands in the sample as compared to the control is an indication of the presence of target nucleic acids of the invention. Alternatively, the target HIF-1α nucleic acids can be identified by sequencing according to well known techniques. In alternative embodiments, oligonucleotide probes specific to the target HIF-1α nucleic acids can be used to detect the presence of specific fragments.

Sequence-specific probe hybridization is a well known method of detecting desired nucleic acids in a sample comprising a biological fluid or tissue sample and is within the scope of the present invention. Briefly, under sufficiently stringent hybridization conditions, the probes hybridize specifically only to substantially complementary sequences. The stringency of the hybridization conditions can be relaxed to tolerate varying amounts of sequence mismatch. If the target is first amplified, detection of the amplified product utilizes this sequence-specific hybridization to insure detection of only the correct amplified target, thereby decreasing the chance of a false positive caused by the presence of homologous sequences from related organisms or other contaminating sequences.

A number of hybridization formats well known in the art, including but not limited to solution phase, solid phase, mixed phase, or in situ hybridization assays are encompassed within the nucleic acid detection methods of the invention. In solution (or liquid) phase hybridizations, both the target nucleic acid and the probe or primer are free to interact in the reaction mixture. In solid phase hybridization assays, either the target or probes are linked to a solid support where they are available for hybridization with complementary nucleic acids in solution. Exemplary solid phase formats include Southern hybridizations, dot blots, and the like. The following articles provide an overview of the various hybridization assay formats, all of which are incorporated herein by reference in their entirety: Singer et al., 1986 Biotechniques 4: 230; Haase et al., 1984, Methods in Virology, Vol. VII, pp. 189-226; Wilkinson, In Situ Hybridization, D. G. Wilkinson ed., IRL Press, Oxford University Press, Oxford; and Nucleic Acid Hybridization: A Practical Approach, Hames, B. D. and Higgins, S. J., eds., IRL Press (1987).

The invention encompasses homogenous based hybridization assays as well as heterogeneous based assays for detection and/or quantitation of HIF-1α nucleic acid sequences in accordance with the methods of the invention. Heterogeneous based assays depend on the ability to separate hybridized from non-hybridized nucleic acids. One such assay involves immobilization of either the target or probe nucleic acid on a solid support so that non-hybridized nucleic acids which remain in the liquid phase can be easily separated after completion of the hybridization reaction (see, e.g., Southern, 1975, J. Mol. Biol. 98: 503-517; which is incorporated herein by reference in its entirety). In comparison, homogeneous assays depend on other means for distinguishing between hybridized and non-hybridized nucleic acids. Because homogeneous assays do not require a separation step, they are generally considered to be more desirable. One such homogeneous assay relies on the use of a label attached to a probe nucleic acid that is only capable of generating a signal when the target is hybridized to the probe (see, e.g., Nelson, et al., 1992, Nonisotopic DNA Probe Techniques, Academic Press, New York, N.Y., pages 274-310; which is incorporated herein by reference in its entirety).

The invention encompasses any method known in the art for enhancing the sensitivity of the detectable signal in such assays, including but not limited to the use of cyclic probe technology (Bakkaoui et al., 1996, BioTechniques 20: 240-8, which is incorporated herein by reference in its entirety); and the use of branched probes (Urdea et al., 1993, Clin. Chem. 39: 725-6; which is incorporated herein by reference in its entirety).

The hybridization complexes are detected according to well known techniques in the art. Nucleic acid probes capable of specifically hybridizing to a target can be labeled by any one of several methods typically used to detect the presence of hybridized nucleic acids. One common method of detection is the use of autoradiography, using probes labeled with ³H, ¹²⁵I, ³⁵S, ¹⁴C, or ³²P, or the like. The choice of radioactive isotope depends on research preferences due to ease of synthesis, stability, and half lives of the selected isotopes. Other labels include compounds (e.g., biotin and digoxigenin), that bind to anti-ligands or antibodies labeled with fluorophores, chemiluminescent agents, or enzymes. Alternatively, probes can be conjugated directly to labels such as fluorophores, chemiluminescent agents or enzymes. The choice of label depends on sensitivity required, ease of conjugation with the probe, stability requirements, and available instrumentation.

The probes and primers of the invention can be synthesized and labeled using techniques known to one skilled in the art. Oligonucleotides for use as probes and primers may be chemically synthesized according to the solid phase phosphoramidite triester method described by Beaucage, S. L. and Caruthers, M. H., 1981, Tetrahedron Lett. 22(20): 1859-1862, using an automated synthesizer, as described in Needham-VanDevanter, D. R., et al. 1984, Nucleic Acids Res. 12: 6159-6168. Purification of oligonucleotides can be by either native acrylamide gel electrophoresis or by anion-exchange HPLC, as described in Pearson, J. D. and Regnier, F. E., 1983, J. Chrom. 255:137-149. All of the references cited supra are incorporated herein by reference in their entirety.

6.3.4 Detection of Proteins

The methods and kits of the invention encompass detection and/or quantitation of HIF-1α in a sample obtained from a subject. Any method known to one skilled in the art for the detection and quantitation of an HIF-1α protein is encompassed within the present invention. HIF-1α protein sequences useful in the methods and kits of the invention are well known in the art. See, for example, Wang et al., 1995, Proc. Natl. Acad. Sci. USA, 92: 5510-4; and WO 96/39426 each of which is incorporated herein by reference in their entireties.

HIF-1α proteins and anti-HIF-1α antibodies and immunospecific fragments thereof are suitable in the assays of the invention. Detection and quantitation of an HIF-1α gene product encompasses the detection of proteins exemplified herein. Detection of reduced levels of an HIF-1α gene product in a sample obtained from a subject in accordance with the methods of the invention is generally compared to a standard sample.

In some embodiments, antibodies directed against naturally occurring HIF-1α proteins may be used in the methods of the invention. The invention encompasses the use of any standard immunoassay method known to one skilled in the art, including but not limited to Western blot, ELISA, and FACS.

In one embodiment, the invention encompasses use of an immunoassay comprising contacting a sample from a subject with an anti-HIF-1α antibody or an immunospecific fragment thereof under conditions such that immunospecific binding to the HIF-1α receptor in the sample can occur, thereby forming an immune complex, and detecting and/or measuring the amount of complex formed. In a specific embodiment, an antibody to an HIF-1α is used to assay a sample for the presence of the HIF-1α, wherein an increased level of the HIF-1α is detected relative to a standard sample.

In some embodiments, the biological sample may be brought in contact with and immobilized onto a solid phase support or a carrier such as nitrocellulose or other solid support capable of immobilizing cells, cell particles or soluble proteins. The support can be washed with suitable buffers followed by treatment with the antibody that selectively or specifically binds to an HIF-1α protein. The solid phase support can then be washed with buffer to remove unbound antibody. The amount of antibody bound to the solid support can then be detected by conventional means.

“Solid phase support or carrier” as used herein refers to any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

In some embodiments, the anti-HIF-1α antibody or an immunospecific fragment thereof can be detectably labeled by linking the same to an enzyme and using the labeled antibody in an enzyme immunoassay (EIA) (Voller, A., “The Enzyme Linked Immunosorbent Assay (ELISA)”, 1978, Diagnostic Horizons 2:1, Microbiological Associates Quarterly Publication, Walkersville, Md.; Voller, A. et al., 1978, J. Clin. Pathol. 31:507-520; Butler, J. E., 1981, Meth. Enzymol. 73:482; Maggio, E. ed., 1980, Enzyme Immunoassay, CRC Press, Boca Raton, Fla.; Ishikawa, E. et al., eds., 1981, Enzyme Immunoassay, Kgaku Shoin, Tokyo, all of which are incorporated herein by reference in their entirety). The enzyme bound to the antibody will react with an appropriate substrate, preferably a chromogenic substrate, in such a manner as to produce a chemical moiety which can be detected, for example, by spectrophotometric, fluorimetric or visual means. Enzymes that can be used to detectably label the antibody include but are not limited to malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase, among others. The detection can be accomplished by colorimetric methods that employ a chromogenic substrate for the enzyme. Detection can also be accomplished by visual comparison of the extent of enzymatic reaction of a substrate in comparison with similarly prepared standards.

Detection can also be accomplished using any other method known to one skilled in the art. For example, by radioactively labeling the antibodies or antibody fragments, it is possible to detect HIF-1α protein through the use of a radioimmunoassay (RIA) (see, for example, Weintraub, B., Principles of Radioimmunoassays, Seventh Training Course on Radioligand Assay Techniques, The Endocrine Society, March, 1986). The radioactive isotope can be detected by such means as the use of a gamma counter or a scintillation counter, or by autoradiography.

In other embodiments, the invention encompasses labeling the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. In yet other embodiments, the antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The invention further encompasses detectably labeling the antibody by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester. Likewise, a bioluminescent compound can be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Bioluminescent compounds for purposes of labeling include, e.g., luciferin, luciferase and aequorin.

The invention also encompasses methods for indirect detection of HIF-1α. In a specific embodiment, the invention encompasses use of an immunoassay comprising contacting a sample derived from a subject with a disease or disorder with an anti-HIF-1α antibody (primary antibody) or an immunospecific fragment thereof under conditions such that immunospecific binding to the HIF-1α protein in the sample can occur, thereby forming an immune complex, adding a secondary antibody that is labeled under conditions such that immunospecific binding to the primary antibody occurs and detecting and/or quantitating the amount of complex formed indirectly.

Anti-HIF-1α antibodies or immunospecific fragments thereof may be used quantitatively or qualitatively to detect an HIF-1α in a sample. In some embodiments, when the sample is a tissue, the anti-HIF-1α antibodies or immunospecific fragments thereof may be used histologically, e.g., immunofluorescence or microscopic studies, using common techniques known to one skilled in the art, for in situ detection of an HIF-1α receptor. In situ detection may be accomplished by preparing a histological specimen from a subject, such as a paraffin embedded section of tissue, e.g., breast tissues, and applying thereto a labeled antibody of the present invention. The antibody (or fragment) is preferably applied by overlaying the labeled antibody (or fragment) onto the biological sample. Through the use of such a procedure, it is possible to determine not only the presence of an HIF-1α protein but also its distribution in the examined tissue. Using the methods of the present invention, those of ordinary skill will readily perceive that any of a wide variety of histological methods (such as staining procedures) can be modified in order to achieve such in situ detection.

6.4 Nucleic Acids Encoding HIF-1 Alpha

The methods of the invention may use any nucleic acid encoding HIF-1α, an analog, fragment or derivative thereof, as a proxy for determining a HIF-1α level. A nucleic acid is intended to include DNA molecules (e.g., cDNA, genomic DNA), RNA molecules (e.g., hnRNA, pre-mRNA, mRNA) and DNA or RNA analogs (e.g., peptide nucleic acids) generated using techniques known to one skilled in the art. The nucleic acid measured as a proxy for a HIF-1α level can be single-stranded or double stranded.

For example, but not by way of limitation, nucleotide sequences for use in the methods and kits of the invention may include all or a portion of any of the following: nucleotide sequences disclosed in U.S. Pat. No. 6,455,674 (issued to Einat et al); U.S. Pat. No. 6,652,799 (issued to Semenza); U.S. Pat. No. 6,222,018(issued to Semenza); Wang et al., 1995 PNAS USA, 92: 5510-4; and WO 96/39426. The invention encompasses all or a portion of the nucleotide sequence of human HIF-1α with GENBANK Accession Nos. NM_{—}001530 and NM_{—}181054. All nucleotide sequences of the references cited supra are incorporated herein by reference in their entirety.

Generally, any HIF-1α nucleic acid known in the art may be useful in the methods and kits of the invention. Such nucleic acids generally encode at least a portion of HIF-1α, or have a sequence that hybridizes to a HIF-1α, -encoding nucleic acid under hybridizing conditions, as described herein.

In one embodiment, the methods of the invention may use a coding sequence or a 5′ or 3′ untranslated region of a nucleic acid encoding HIF-1α or a fragment thereof as a probe, including naturally occurring and non-naturally occurring variants. A non-naturally occurring variant is one that is engineered by man (e.g., a peptide nucleic acid probe). In the methods of the invention wherein HIF-1α, or an mRNA encoding HIF-1α, in a sample from a subject is detected or measured, naturally occurring gene products are detected, including but not limited to wild-type gene products as well as mutants, allelic variants, splice variants, polymorphic variants, etc. In general, variants will be highly homologous to the wild-type gene product encoding HIF-1α, e.g., having at least 90%, 95%, 98% or 99% amino acid sequence identity (as determined by standard algorithms known in the art, see, e.g., Altschul, 1990 Proc. Natl. Acad. Sci. U.S.A. 87: 2264-2268; Altschul, 1993, Proc. Natl. Acad. Sci. U.S.A. 90: 5873-5877; Altschul et al., 1990 J. Mol. Biol. 215: 403-410).

HIF-1α variants to be used as probes may be encoded by a nucleic acid which is hybridizable under stringent conditions to a nucleic acid encoding HIF-1α. Nucleic acid hybridization methods are well known in the art (see, e.g., Sambrook et al., 2001 Molecular Cloning, A Laboratory Manual, 3^{rd} ed., Cold Spring Harbor Laboratory Press, Cold Spring Harbor, N.Y.; Ausubel et al., eds., 1994-1997, in the Current Protocols in Molecular Biology: Series of laboratory technique manuals, John Wiley and Sons, Inc.; Shilo and Weinberg, 1981, Proc. Natl. Acad. Sci. U.S.A. 78, 6789-92; Dyson, 1991 Essential Molecular Biology: A Practical Approach, vol. 2, T. A. Brown, ed., 111-156, Press at Oxford University Press, Oxford, UK). The term “stringent conditions” refers to the ability of a first polynucleotide molecule to hybridize, and remain bound to a second filter-bound polynucleotide molecule in 0.5 M NaHPO₄, 7% sodium dodecyl sulfate (SDS), 1 mM EDTA at 65° C., followed by washing in 0.2×SSC/0.1% SDS at 42° C. (see Ausubel et al. (eds.), 1989, Current Protocols in Molecular Biology, Vol. 1, Green Publishing Associates, Inc., and John Wiley & Sons, Inc., New York, at p. 2.10.3). In specific embodiments, the variants being detected or measured comprise (or, if nucleic acids, encode) not more than 1, 2, 3, 4, 5, 10, 15 or 20 point mutations (substitutions) relative the wild-type sequence.

An isolated nucleic acid probe encoding HIF-1α or a portion thereof, can be obtained by any method known in the art, e.g., from a deposited plasmid, by PCR amplification using synthetic primers hybridizable to the 3′ and 5′ ends of the sequence, and/or by cloning from a cDNA or genomic library using standard screening techniques, or by polynucleotide synthesis. Use of such probes for detection and quantitation of specific sequences is well known in the art. See e.g., Erlich, e.d., 1989, PCR Technology Principles and Applications for DNA Amplification, Macmillan Publishers Ltd., England; Sambrook et al, Molecular Cloning: A Laboratory Manual, 3^{rd} ed., Cold Spring Harbor Laboratory Press, Cold Spring Harbor, N.Y., 2001.

In some embodiments, the methods of the invention may use a gene coding sequence, e.g., cDNA, of HIF-1α, which preferably hybridizes under stringent conditions as described above to at least about 6, preferably about 12, most preferably about 18 or more consecutive nucleotides of the gene coding sequence of HIF-1α protein, useful for the detection of an HIF-1α protein

Using all or a portion of a nucleic acid sequence encoding HIF-1α protein, such as those exemplified herein as a hybridization probe, full length nucleic acid molecules encoding HIF-1α protein can be quantitated using standard hybridization techniques (see, e.g., Sambrook et al., Molecular Cloning: A Laboratory Manual, 3^{rd} ed., Cold Spring Harbor Laboratory, Cold Spring Harbor Laboratory Press, Cold Spring Harbor, N.Y., 2001) for use in the methods of the invention, i.e., as a proxy for HIF-1α level.

The HIF-1α sequences used in the methods of the invention are preferably human sequences. However, homologs of the human HIF-1α isolated from other animals can also be used in the methods of the invention as a proxy for HIF-1α level, particularly where the subject is a non-human animal. Thus, the invention also includes the use of HIF-1α homologs identified from non-human animals such as non-human primates, rats, mice, farm animals including but not limited to cattle, horses, goats, sheep, pigs, etc., household pets including but not limited to cats, dogs, etc., in the methods of the invention.

The methods of the invention may use fragments of any of the nucleic acids disclosed herein in any of the methods of the invention. A fragment preferably comprises at least 10, 20, 50, 100, or 200 contiguous nucleotides of a sequence described herein.

Standard recombinant DNA techniques known in the art may be used to provide a HIF-1α protein or a nucleic acid encoding an HIF-1α protein, or a fragment thereof, for use in the methods and kits of the invention. In some embodiments, in order to provide a HIF-1α or nucleic acid as a standard, the corresponding nucleotide sequence encoding HIF-1α protein of interest can be cloned. For a review of PCR technology and cloning strategies which may be used in accordance with the invention, see, e.g., PCR Primer, 1995, Dieffenbach et al., ed., Cold Spring Harbor Laboratory Press; Sambrook et al., 2001, supra which are incorporated herein by reference in their entireties.

6.5 HIF-1 Alpha Proteins

The present invention provides for the use of HIF-1α proteins, or fragments thereof, for the generation of antibodies for methods of the invention. HIF-1α polypeptides and fragments can also be used as protein abundance or activity standards in the methods of the invention.

For example, but not by way of limitation, the invention encompasses amino acid sequences of HIF-1α as disclosed in U.S. Pat. No. 6,455,674 (issued to Einat et al); U.S. Pat. No. 6,652,799 (issued to Semenza); U.S. Pat. No. 6,222,018(issued to Semenza); Wang et al., 1995 PNAS USA, 92: 5510-4; U.S. Pat. No. 6,436,654 (issued to Berkenstam); WO 96/39426. The amino acid sequences cited in the above-identified references are incorporated herein by reference in their entirety. The invention encompasses the amino acid sequences of human HIF-1α with GENBANK Accession No.s NP_{—}001521 and NP_{—}851397 each of which is incorporated herein by reference in its entirety.

In some embodiments, the HIF-1α protein comprises an amino acid sequence that exhibits at least about 65%, 70%, 75%, 80%, 85%, 90%, 95%, or 99% sequence similarity to the amino acid sequence of any of HIF-1α proteins known in the art. Algorithms for determining percent identity between two protein sequences are well known in the art, see, e.g., Altschul, 1990 Proc. Natl. Acad. Sci. U.S.A. 87: 2264-2268; Altschul, 1993, Proc. Natl. Acad. Sci. U.S.A. 90: 5873-5877; Altschul et al., 1990 J. Mol. Biol. 215: 403-410; each of which is incorporated herein by reference.

In a specific embodiment, proteins are provided consisting of or comprising a fragment of HIF-1α protein consisting of at least ten contiguous amino acids. In another embodiment, the fragment consists of or comprises at least 20, 30, 40, or 50 contiguous amino acids from a HIF-1α protein for use, for example, in raising antibodies. Such fragments can also be useful, for example, as standards or controls in the methods and kits of the invention.

A variety of host-expression vector systems may be utilized to express HIF-1α proteins or fragments for use in the methods of the invention. Such host-expression systems are well known and provide the necessary means by which a protein of interest may be produced and subsequently purified. Examples of host-expression vector systems that may be used in accordance with the invention are: bacterial cells (e.g., E. coli, B. subtilis) transformed with recombinant bacteriophage DNA, plasmid DNA or cosmid DNA expression vectors containing a HIF-1α nucleic acid coding sequence, yeast cells (e.g., Saccharomyces, Pichia) transformed with a recombinant yeast expression vector containing the HIF-1α coding sequence; insect cells infected with a recombinant virus expression vector (e.g., baculovirus) containing the HIF-1α coding sequence; plant cells infected with a recombinant virus expression vector (e.g., cauliflower mosaic virus, CaMV; tobacco mosaic virus, TMV) or transformed with a recombinant plasmid expression vector (e.g., Ti plasmid) containing the HIF-1α coding sequence; or mammalian cells (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the HIF-1α being expressed. For example, when a large quantity of such a protein is to be produced for raising antibodies, vectors that direct the expression of high levels of protein products that are readily purified may be desirable. Such vectors include but are not limited to the E. coli expression vector pUR278 (Ruther et al., 1983, EMBO J. 2:1791), in which the HIF-1α coding sequence can be ligated into the vector in-frame with the lac Z coding region so that a fusion protein is produced; pIN vectors (Inouye & Inouye, 1985, Nucleic Acids Res. 13:3101; Van Heeke & Schuster, 1989, J. Biol. Chem. 264:5503); and the like. pGEX vectors can also be used to express foreign polypeptides as fusion proteins with glutathione S-transferase (GST). In general, such fusion proteins are soluble and can easily be purified from lysed cells by adsorption and binding to a column comprising of glutathione-agarose beads followed by elution in the presence of free glutathione. The pGEX vectors are designed to include, e.g., thrombin or factor Xa protease cleavage sites so that the cloned target gene product can be released from the GST moiety.

In an insect system, Autographa californica nuclear polyhedrosis virus (AcNPV) can be used as a vector to express foreign genes. The virus grows in Spodoptera frugiperda cells. The HIF-1α coding sequence can be cloned individually into non-essential regions (for example the polyhedrin gene) of the virus and placed under control of an AcNPV promoter (for example the polyhedrin promoter). Successful insertion of a HIF-1α coding sequence will result in inactivation of the polyhedrin gene and production of non-occluded recombinant virus (i.e., virus lacking the proteinaceous coat coded for by the polyhedrin gene). These recombinant viruses can be used to infect Spodoptera frugiperda cells in which the inserted gene is expressed (e.g., see Smith et al., 1983, J. Virol. 46:584; Smith, U.S. Pat. No. 4,215,051).

In mammalian host cells, a number of viral-based expression systems can be utilized. In cases where an adenovirus is used as an expression vector, the HIF-1α coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing HIF-1α in infected hosts (see, e.g., Logan & Shenk, 1984, Proc. Natl. Acad. Sci. USA 81:3655). Specific initiation signals may also be required for efficient translation of inserted HIF-1α coding sequences. These signals include the ATG initiation codon and adjacent sequences. In cases where an entire HIF-1α gene, including its own initiation codon and adjacent sequences, is inserted into the appropriate expression vector, no additional translational control signals may be needed. However, in cases where only a portion of the HIF-1α coding sequence is inserted, exogenous translational control signals, including, if necessary, the ATG initiation codon, must be provided. These exogenous translational control signals and initiation codons can be of a variety of origins, both natural and synthetic. Furthermore, the initiation codon must be in phase with the reading frame of the desired coding sequence to ensure correct translation of the entire insert. The efficiency of expression may be enhanced by the inclusion of appropriate transcription enhancer elements, transcription terminators, etc. (see Bittner et al., 1987, Methods in Enzymol. 153: 516).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product can be used. Such mammalian host cells include but are not limited to CHO, VERO, BHK, HeLa, COS, MDCK, 293, 3T3, W138, and in particular, breast cancer cell lines such as, for example, BT483, Hs578T, HTB26, BT20 and T47D, and normal mammary gland cell lines such as, for example, CRL7030 and Hs578Bst.

For long-term, high-yield production of recombinant proteins, stable expression is preferred. For example, cell lines that stably express the HIF-1α gene product can be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker.

Following introduction of the foreign DNA, engineered cells can be allowed to grow for 1-2 days in an enriched media, and then can be switched to a selective media. A selectable marker in a recombinant construct, such as a plasmid, can confer resistance to the selective media, allow cells to stably integrate the plasmid into their chromosomes, and grow to form foci which, in turn, can be cloned and expanded into cell lines. This method can advantageously be used to engineer cell lines that stably express the HIF-1α gene product. Such engineered cell lines can be particularly useful in screening and evaluating compounds that affect the endogenous activity of the HIF-1α gene product.

A number of selection systems including but not limited to the herpes simplex virus thymidine kinase (Wigler et al., 1977, Cell 11: 223), hypoxanthine-guanine phosphoribosyltransferase (Szybalska & Szybalski, 1962, Proc. Natl. Acad. Sci. USA 48: 2026), and adenine phosphoribosyltransferase (Lowy et al., 1980, Cell 22: 817) genes can be employed in tk⁻, hgprt⁻ or aprt⁻ cells, respectively. Also, anti-metabolite resistance can be used as the basis of selection for the following genes: dhfr, which confers resistance to methotrexate (Wigler et al., 1980, Proc Natl. Acad. Sci. USA 77: 3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150: 1); and hygro, which confers resistance to hygromycin (Santerre et al., 1984, Gene 30: 147).

6.6 Antibodies to HIF-1 Alpha

The methods and kits of the invention encompass use of anti-HIF-1α antibodies or fragments thereof that specifically recognize one or more epitopes of a HIF-1α protein. Accordingly, any HIF-1α protein, derivative, or fragment can be used as an immunogen to generate antibodies that immunospecifally bind a HIF-1α protein. Such antibodies and fragments can be used in the detection and quantitation of a HIF-1α in a sample to carry out any of the methods of the invention as disclosed herein.

Such antibodies can include but are not limited to polyclonal antibodies, monoclonal antibodies (mAbs), humanized or chimeric antibodies, single chain antibodies, Fab fragments, F(ab′)₂ fragments, Fv fragments, fragments produced by a Fab expression library, anti-idiotypic (anti-Id) antibodies, and epitope-binding fragments of any of the above. In a specific embodiment, antibodies to human HIF-1α protein are used.

Described herein are general methods for the production of antibodies or immunospecific fragments thereof. Any of such antibodies or fragments can be produced by standard immunological methods or by recombinant expression of nucleic acid molecules encoding the antibody or an immunospecific fragment thereof in an appropriate host organism.

For the production of antibodies against HIF-1α, any of various host animals can be immunized by injection with a HIF-1α gene product, or a portion thereof. Such host animals can include but are not limited to rabbits, mice, and rats. Various adjuvants can be used to increase the immunological response depending on the host species, including but not limited to Freund's (complete or incomplete), mineral gels such as aluminum hydroxide, surface active substances such as lysolecithin, pluronic polyols, polyanions, peptides, oil emulsions, keyhole limpet hemocyanin, dinitrophenol or potentially useful human adjuvants such as BCG (bacille Calmette-Guerin) and Corynebacterium parvum.

Anti-HIF-1α monoclonal antibodies are preferred for use in the methods and kits of the invention. Monoclonal antibodies can be obtained by any technique that provides for the production of antibody molecules by continuous cell lines in culture. These include but are not limited to the hybridoma technique of Kohler and Milstein, (1975, Nature 256: 495; and U.S. Pat. No. 4,376,110), the human B-cell hybridoma technique (Kosbor et al., 1983, Immunology Today 4: 72; Cole et al., 1983, Proc. Natl. Acad. Sci. USA 80: 2026), and the EBV-hybridoma technique (Cole et al., 1985, Monoclonal Antibodies And Cancer Therapy, Alan R. Liss, Inc., pp. 77). Such antibodies can be of any immunoglobulin class, including IgG, IgM, IgE, IgA, IgD, and any subclass thereof. The hybridoma producing the mAb of this invention can be cultivated in vitro or in vivo.

Techniques developed for the production of “chimeric antibodies” (Morrison et al., 1984, Proc. Natl. Acad. Sci. 81, 6851-6855; Neuberger et al., 1984, Nature 312, 604-608; Takeda et al., 1985, Nature 314, 452-454) by splicing the genes from a mouse antibody molecule of appropriate antigen specificity together with genes from a human antibody molecule of appropriate biological activity can be used in preparing antibodies useful in the present invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 5,816,397). The invention thus contemplates chimeric antibodies that are specific or selective for a HIF-1α protein. While often designed to be therapeutic, such chimeric antibodies can be useful to quantitate a HIF-1α level according to the methods of the invention.

Further, humanized antibodies can be used in the methods and kits of the invention. Briefly, humanized antibodies are antibody molecules from non-human species having one or more hypervariable regions or complementarity determining regions (CDRs) from the non-human species and framework regions from a human immunoglobulin molecule. The extent of the framework region and Cars have been precisely defined (see, “Sequences of Proteins of Immunological Interest”, Kabat, E. et al., U.S. Department of Health and Human Services (1983)). Examples of techniques that have been developed for the production of humanized antibodies are known in the art and useful within the scope of the present invention. (See, e.g., Queen, U.S. Pat. No. 5,585,089 and Winter, U.S. Pat. No. 5,225,539). Humanized antibodies are typically developed as therapeutic agents. However, such antibodies can also be used in the methods and kits of the present invention, as they can be used to quantitate a HIF-1α level in accordance with the instant invention.

Phage display technology can be used to increase the affinity of an antibody to a HIF-1α gene product. This technique can be useful in obtaining higher affinity antibodies to an HIF-1α gene product, which could be used for the diagnosis and prognosis of a subject with a disease or disorder according to the present invention. The technology, referred to as affinity maturation, employs mutagenesis or CDR walking and re-selection using the HIF-1α gene product antigen to identify antibodies that bind with higher affinity to the antigen when compared with the initial or parental antibody (see, e.g., Glaser et al., 1992, J. Immunology 149:3903). Mutagenizing entire codons rather than single nucleotides results in a semi-randomized repertoire of amino acid mutations. Libraries can be constructed consisting of a pool of variant clones, each of which differs by a single amino acid alteration in a single CDR, and contain variants representing each possible amino acid substitution for each CDR residue. Mutants with increased binding affinity for the antigen can be screened by contacting the immobilized mutants with labeled antigen. Any screening method known in the art can be used to identify mutant antibodies having increased avidity to the antigen (e.g., ELISA) (see Wu et al., 1998, Proc Natl. Acad. Sci. USA 95:6037; Yelton et al., 1995, J. Immunology 155:1994). CDR walking that randomizes the light chain may also be useful (see Schier et al., 1996, J. Mol. Bio. 263:551).

Alternatively, techniques described for the production of single chain antibodies (U.S. Pat. No. 4,946,778; Bird, 1988, Science 242:423; Huston et al., 1988, Proc. Natl. Acad. Sci. USA 85:5879; and Ward et al., 1989, Nature 334: 544) can be adapted to produce single chain antibodies against HIF-1α gene products. Single chain antibodies are formed by linking the heavy and light chain fragments of the Fv region via an amino acid bridge, resulting in a single chain polypeptide. Techniques for the assembly of functional Fv fragments in E. coli can also be used (Skerra et al., 1988, Science 242:1038).

Antibody fragments that recognize specific epitopes can be generated by known techniques. Such fragments can be used for quantitating a HIF-1α gene product according to any available method known in the art. For example, such fragments include but are not limited to: F(ab′)₂ fragments, which can be produced by pepsin digestion of the antibody molecule; and Fab fragments, which can be generated by reducing the disulfide bridges of the F(ab′)₂ fragments; Fab fragments, which can be generated by treating the antibody molecule with papain and a reducing agent; and Fv fragments. Alternatively, Fab expression libraries can be constructed (Huse et al., 1989, Science 246:1275-1281) to allow rapid and easy identification of monoclonal Fab fragments having the desired specificity.

A molecular clone of an antibody to an antigen of interest can be prepared by techniques known to one skilled in the art. Recombinant DNA methodology (see e.g., Maniatis et al., 1982, Molecular Cloning. A Laboratory Manual, Cold Spring Harbor Laboratory, Cold Spring Harbor, N.Y.) can be used to construct nucleic acid sequences that encode a monoclonal antibody molecule, or an immunospecific fragment thereof.

Antibody molecules can be purified by well-known techniques, e.g., immunoabsorption or immunoaffinity chromatography, chromatographic methods such as HPLC (high performance liquid chromatography), or a combination thereof.

In the production of antibodies, screening for the desired antibody can be accomplished by techniques known in the art, e.g., ELISA (enzyme-linked immunosorbent assay). For example, to select antibodies that recognize a specific domain of an HIF-1α, generated hybridomas can be assayed for a product that binds to a HIF-1α fragment containing such domain.

The foregoing antibodies can be used to quantify a HIF-1α protein, e.g., to measure levels thereof in appropriate samples, in the methods and kits of the invention.

The methods of antibody production employed herein include those described in Harlow and Lane (Harlow, E. and Lane, D., 1988, and later editions, Antibodies: A Laboratory Manual, Cold Spring Harbor Laboratory Press, Cold Spring Harbor, N.Y.), which is incorporated herein by reference in its entirety.

Any antibody directed to one or more epitopes of an HIF-1α can be used in the methods and kits of the invention. Commercially available HIF-1α antibodies can be used in accordance with the instant invention, for example those available from Novus Biologicals, Inc. (Littleton, Colo.); Affinity BioReagents, (Golden, Colo.).

6.7 Kits

The invention provides a pharmaceutical pack or kit comprising one or more containers filled with compounds of the invention. Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. The present invention provides kits that can be used in the above methods. In one embodiment, a kit comprises one or more compounds of the invention. In another embodiment, a kit further comprises one or more other prophylactic or therapeutic agents useful for the treatment of ischemia, in one or more containers.

### 7. EXAMPLES

The following examples illustrate aspects of this invention but should not be construed as limitations. The symbols and conventions used in these examples are intended to be consistent with those used in the contemporary, international, chemical literature, for example, the Journal of the American Chemical Society (J. Am. Chem. Soc.) and Tetrahedron.

7.1 Regulation of HIF-1A by Adenosine

Chemicals and Reagents: A375 melanoma, NCTC 2544 keratinocytes, U2OS osteosarcoma, U87MG glioblastoma human cells were obtained from American Tissue Culture Collection (ATCC). Tissue culture media and growth supplements were obtained from BioWhittaker. GasPak Pouch™ System was obtained from Becton Dickinson. Unless otherwise noted, all other chemicals were purchased from Sigma. Anti-HIF-1α and anti-HIF1β antibodies (mAb) were obtained from Transduction Laboratories (BD, Milano, Italy). U0126 (inhibitor of MEK-1 and MEK-2), SB202190 (inhibitor of p38 MAP kinase), Anti-ACTIVE®MAPK and anti-ERK 1/2 (pAb) were from Promega. Phospho-p38 and p38 MAP Kinase antibodies were from Cell Signaling Technology. Anti-Adenosine A₃ receptor (polyAb) was from Aviva Antibody Corporation.

Cell culture and hypoxia treatment: Cells were maintained in DMEM (A375), EMEM (NCTC 2544) or RPMI 1640 (U87MG, U2OS) medium containing 10% fetal calf serum, penicillin (100 U/ml), streptomycin (100 ug/ml), and L-glutamine (2 mM) at 37° C. in 5% CO₂/95% air. Cells were passaged two or three times weekly at a ratio between 1:5 and 1:10. Hypoxic exposure was in BBL™ GasPak pouch™ System (Becton Dickinson) that reduce oxygen concentration of less than 2% within 2 hours of incubation at 35° C.

[³H]-Thymidine incorporation: cell proliferation test: Cells were seeded in fresh medium with 1 uCi/ml of [³H]-Thymidine in DMEM containing 10% fetal calf serum, penicillin (100 U/ml), streptomycin (100 ug/ml), L-glutamine (2 mM). After 24 hours of labelling, cells were trypsinised, dispensed in 4 wells of a 96 well plate, and filtered through Whatman GF/C glass-fiber filters using a Micro-Mate 196 cell harvester (Packard Instrument Company). The filter bound radioactivity was counted on Top Count Microplate Scintillation Counter (efficiency 57%) with Micro-Scint 20.

Flow Cytometry analysis: A375 adherent cells were trypsinized, mixed with floating cells, washed with PBS and permeabilized in 70% (vol/vol) ethanol/PBS solution at 4° C. for at least 24 hours. The cells were washed with PBS and the DNA was stained with a PBS solution, containing 20 ug/ml of propidium iodide and 100 ug/ml of RNAse, at room temperature for 30 minutes. Cells were analysed with an EPICS XL flow cytometer (Beckman Coulter, Miami, Fla.) and the content of DNA was evaluated by the Cell-LISYS program (Becton-Dickinson). Cell distribution among cell cycle phases and the percentage of apoptotic cells were evaluated as previously described (Merighi 2002). Briefly, the cell cycle distribution is shown as the percentage of cells containing 2n (G₀/G₁ phases), 4n (G₂ and M phases), 4n>x>2n DNA amount (S phase) judged by propidium iodide staining. The apoptotic population is the percentage of cells with DNA content lower than 2n.

Small interfering RNA (siRNA) design: To generate a small interfering RNA that targets A₃ receptor mRNA (siRNA_{A3}), eight oligonucleotides consisting of ribonucleosides, except for the presence of 2′-deoxyribonucleosides at the 3′ end, were synthesized and annealed, according to the recommendations of Elbashir (ref), to the manufacturer's instructions (Silencer™ siRNA Construction Kit, Ambion) and as previously described (Mirandola, 2004). For oligo-1, sense sequence: 5′-GCU UAC CGU CAG AUA CAA GUU-3′ (SEQ ID NO:1) and antisense 5′-CUU GUA UCU GAC GGU AAG CUU-3′ (SEQ ID NO:2). For oligo-2, sense sequence: 5′-GAC GGC UAA GUC CUU GUU UUU-3′ (SEQ ID NO:3) and antisense 5′-AAA CAA GGA CUU AGC CGU CUU-3′ (SEQ ID NO:4). For oligo-3, sense sequence: 5′-ACA CUU GAG GGC CUG UAU GUU-3′ (SEQ ID NO:5) and antisense 5′-CAU ACA GGC CCU CAA GUG UUU-3′ (SEQ ID NO:6). For oligo-4, sense sequence 5′-CCU GCU CUC GGA GGA UGC CUU-3′ (SEQ ID NO:7) and antisense 5′-GGC AUC CUC CGA GAG CAG GUU-3′ (SEQ ID NO:8). Target sequences were aligned to the human genome database in a BLAST search to ensure sequences without significant homology to other genes. The target sequences for oligo-1, oligo-2, oligo-3 and oligo-4 are localized at position 337, 679, 1009 and 1356 bases downstream of the start codon of A₃ receptor mRNA sequence (L20463), respectively.

Treatment of cells with siRNA: A375 cells were plated in six-well plates and grown to 50-70% confluence before transfection. Transfection of siRNA was performed at a concentration of 100 nM using RNAiFect™ Transfection Kit (Qiagen). Cells were cultured in complete media and at 24, 48 and 72 hours total RNA was isolated for Real-Time RT-PCR analysis of A₃ receptor mRNA and for Western blot analysis of A₃ receptor protein. At 48 hours from the transfection, A375 cells were serum starved for another 24 hours and then exposed to increasing concentrations of the A₃ adenosine receptor agonist CL-IB-MECA for 4 hours in hypoxia. Total protein were then harvested for Western blot analysis. As control, cells were exposed to RNAiFect™ Transfection reagent without siRNA_{A3}. To quantify cell transfection efficiency we used siRNA-FITC labelled (Qiagen). After 24 hours of transfection, cells were tripsinized and resuspended in PBS for flow cytometry analysis. Fluorescence obtained from FITC-siRNA transfected cells was compared to autofluorescence generated by untransfected control.

Real-Time RT-PCR experiments: Total cytoplasmic RNA was extracted by the acid guanidinium thiocyanate phenol method (Chomczynski & Sacchi, 1987). Quantitative real-time RT-PCR assay (Higuchi, 1993) of HIF-1α and A₃ mRNA transcripts was carried out using gene-specific double fluorescently labelled TaqMan MGB probe (minor groove binder) in a ABI Prism 7700 Sequence Detection System (Applied Biosystems, Warrington Cheshire, UK). The following primer and probe sequences were used for real-time RT-PCR: A₃ forward primer, 5′-ATG CCT TTG GCC ATT GTT G-3′ (SEQ ID NO:9); A₃ reverse primer, 5′-ACA ATC CAC TTC TAC AGC TGC CT-3′ (SEQ ID NO:10); A₃ MGB probe, 5′-FAM-TCA GCC TGG GCA TC-TAMRA-3′ (SEQ ID NO:11); for the real-time RT-PCR of the HIF-1α gene the assays-on-demand™ Gene expression Product Accession No. NM 019058 was used (Applied Biosystems, Monza, Italy). The fluorescent reporter FAM and the quencher TAMRA are 6-carboxy fluorescein and 6-carboxy-N,N,N′,N′-tetramethylrhodamine, respectively. For the real-time RT-PCR of the reference gene the endogenous control human β-actin kit was used, and the probe was fluorescent-labeled with VIC™ (Applied Biosystems, Monza, Italy).

Western blotting: A375, NCTC 2544, U2OS and U87MG cells were treated with adenosine or adenosine analogues and exposed to normoxia and hypoxia for different times (2-24 hours). Cells were harvested and washed with ice-cold PBS containing 1 mM sodium orthovanadate, AEBSF 104 mM, aprotinin 0.08 mM, leupeptin 2 mM, bestatin 4 mM, pepstatin A 1.5 mM, E-64 1.4 mM. Cells were then lysed in Triton lysis buffer. The protein concentration was determined using BCA protein assay kit (Pierce). Equivalent amounts of protein (35 ug) were subjected to electrophoresis on 7.5% sodium dodecyl sulfate-acrylamide gel. The gel was then electroblotted onto a nitrocellulose membrane. Membranes were blocked with 5% nonfat dry milk in PBS containing 0.1% Tween-20 and incubated with antibodies against HIF-1α (1:250 dilution) and HIF-1β (1:1000 dilution) in 5% nonfat dry milk in PBS/0.1% Tween-20 overnight at 4° C. Aliquots of total protein sample (50 μg) were analyzed using antibodies specific for phosphorylated (Thr183/Tyr185) or total p44/p42 MAPK (1:5000 dilution), phosphorylated (Thr180/Tyr182) or total p38 MAPK (1:1000 dilution) and for A₃ receptor (1 μg/ml dilution). Filters were washed and incubated for 1 hour at room temperature with peroxidase-conjugated secondary antibodies against mouse and rabbit IgG (1:2000 dilution). Specific reactions were revealed with the Enhanced Chemiluminescence Western blotting detection reagent (Amersham Corp., Arlington Heights, Ill.). The membranes were then stripped and reprobed with tubulin (1:250) to ensure equal protein loading.

Metabolic inhibitors: Cells were treated for 30 minutes with metabolic inhibitors or with drug vehicle (DMSO) prior to being challenged with adenosine or adenosine analogues. U0126 was used at 10 and 30 uM as inhibitor of MEK-1 and MEK-2 to prevent p44 and p42 MAPK activation. SB202190 was used at 1 and 10 μM as inhibitor of p38 MAPK.

Densitometry analysis: The intensity of each band in immunoblot assay was quantified using molecular analyst/PC densitometry software (Bio-Rad). Mean densitometry data from independent experiments were normalized to result in cells in the control. The data were presented as the mean±S.E., and analyzed by the Student's test.

Statistical analysis: All values in the figures and text are expressed as mean±standard error (S.E.) of n observation (with n≧3). Data sets were examined by analysis of variance (ANOVA) and Dunnett's test (when required). A P value less than 0.05 was considered statistically significant.

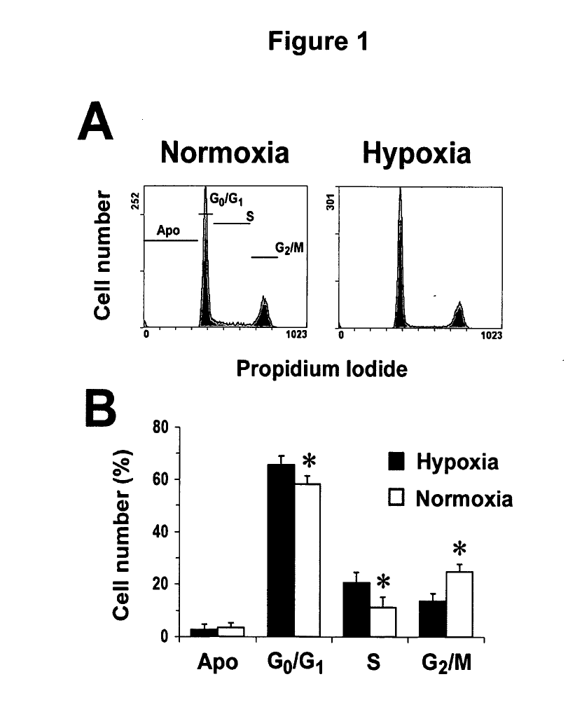

Results: Adenosine induces HIF-1α protein accumulation in hypoxia. We have evaluated the biological effect produced by a prolonged oxygen deprivation in the human A375 melanoma cell line. Viability and proliferation of A375 cells exposed to hypoxia for 24 hours were assessed analyzing the percentage of apoptotic cells and the distribution among the different phases of the cell cycle. We employed flow cytometry and DNA staining by propidium iodide for discrimination of cells in apoptosis, in G₀/G₁, S and G₂/M phases. The results indicate that hypoxia did not promote significant cell death while interfered with proliferation arresting melanoma cells in G₀/G₁ and S phases and reducing the number of cells in G₂/M (FIG. 1). These data were confirmed by using trypan blue exclusion, cell counts and [³H]-thymidine incorporation assay (data not shown).

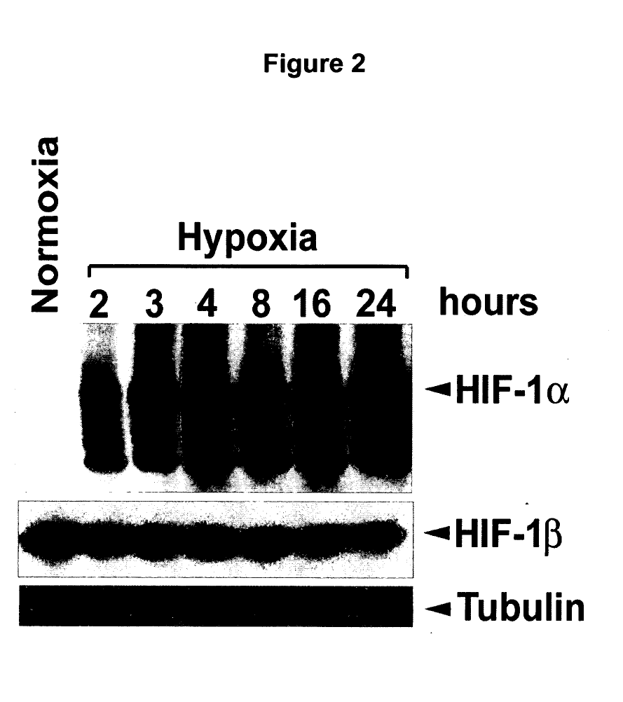

Exposure of A375 cells to hypoxia induced HIF-1α protein expression (FIG. 2). The hypoxic induction of HIF-1α was rapid, and an increase could be seen over the first 2 to 3 hours following hypoxic incubation. Maximal stimulation was obtained between 4-8 hours of incubation in oxygen-deprived conditions, while the levels of HIF-1α protein were somewhat lower with prolonged hypoxia. On the contrary, the levels of HIF-1β were not altered.

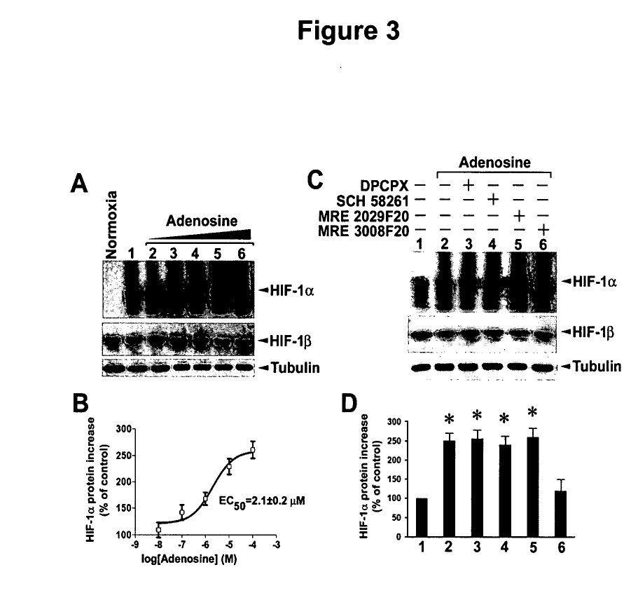

To study the effect of adenosine on the transcription factor HIF-1, A375 melanoma cells were treated for 4 hours with increasing concentrations of the nucleoside in hypoxic conditions. As observed in FIG. 3A, adenosine up-regulated HIF-1α protein expression in hypoxic melanoma cells. In particular, adenosine induced HIF-1α protein accumulation in a dose-dependent manner, with an EC₅₀=2.1±0.2 μM and a maximal increase of 2.6±0.2 fold at 100 μM (FIG. 3B). We did not observe any modulation of HIF-1β protein.

The family of adenosine receptors consists of four subtypes of G protein-coupled receptors, designed A₁, A_{2A}, A_{2B} and A₃. We have previously demonstrated that all four adenosine receptors are expressed in human melanoma A375 cells. To evaluate the functional role of adenosine receptor subtypes on HIF-1α protein expression under hypoxic conditions, we tested the effect of adenosine in combination with DPCPX (an A₁ receptor antagonist), SCH 58261 (a selective A_{2A} receptor antagonist), MRE 2029F20 (a selective A_{2B} receptor antagonist), and MRE 3008F20 (a selective A₃ receptor antagonist) (Baraldi 2004; Merighi 2001; Varani 2000). While the A₁, A_{2A} and A_{2B} receptor antagonists were not able to prevent adenosine-induced HIF-1α protein expression, the A₃ receptor antagonist MRE 3008F20 abrogated the adenosine-induced increase of HIF-1α protein expression (FIG. 3C-D). Furthermore, HIF-1β expression was unaffected by adenosine or by synthetic adenosine receptor antagonists. These results indicate that adenosine may increase HIF-1α protein expression via A₃ receptors.

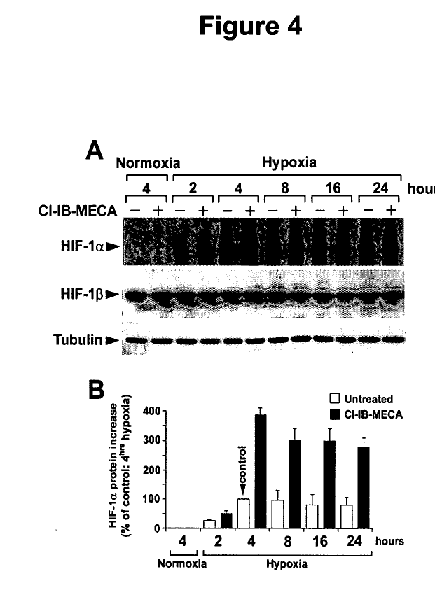

A₃ adenosine receptor induces HIF-1α protein accumulation in hypoxia. To investigate the involvement of A₃ receptors in the modulation of HIF-1α protein expression, we treated A375 cells with the selective A₃ receptor agonist Cl-IB-MECA. We performed a time-course experiment in which A375 cells were exposed to Cl-IB-MECA 100 nM for 2-24 hours. A₃ adenosine receptor stimulation did not promote HIF-1α protein accumulation in normoxia, while under hypoxic conditions HIF-1α protein expression was increased in a time-dependent manner (FIG. 4). In particular, HIF-1α increased from 2 hours and maximum peak levels were observed 4 hours after the addition of Cl-IB-MECA to the culture media. The prolonged A₃ receptor stimulation in hypoxia resulted in a minor effect in HIF-1α protein level modulation. As already observed with adenosine, also Cl-IB-MECA did not modify HIF-1β expression in normoxia and in hypoxia.

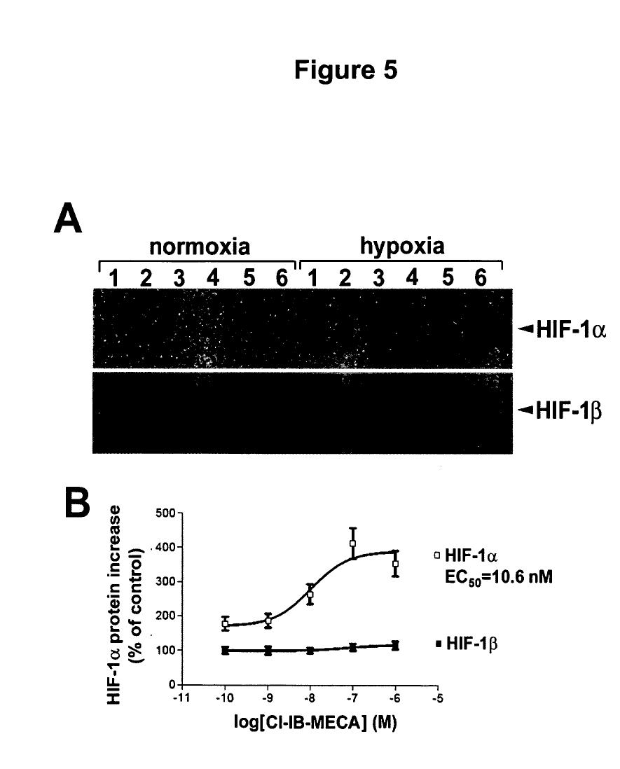

To characterize in more detail the induction of HIF-1α expression by A₃ receptor stimulation, A375 cells were treated with various concentrations of A₃ agonist for 4 hours. As expected, in normoxia the activation of A₃ receptors did not induce detectable levels of HIF-1α. On the contrary, in hypoxia Cl-IB-MECA induced HIF-1α protein accumulation in a dose-dependent manner (FIG. 5A), reproducing the effect produced by adenosine (FIG. 3). The maximum expression of HIF-1α protein was induced by Cl-IB-MECA at a concentration of 100 nM, with an EC₅₀=10.6+1.2 nM (FIG. 5B). In contrast, A₃ receptor stimulation did not affect the expression of HIF-1]protein, either in normoxia or in hypoxia.

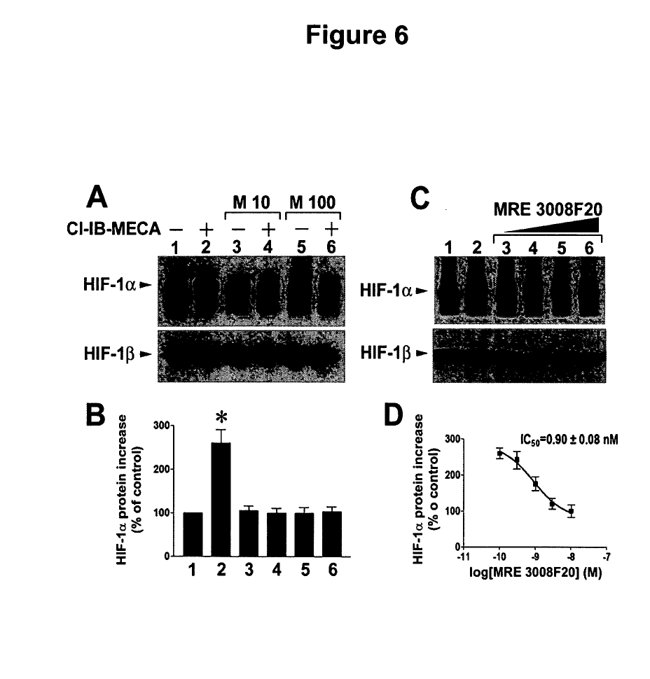

To better characterize the ability of A₃ receptor to significantly increase HIF-1α protein expression in hypoxia, we performed a series of experiments to evaluate the ability of A₃ selective receptor antagonists (MRE 3008F20 and MRE 3005F20) (Baraldi 2004) to prevent this effect. A375 cells were treated with increasing concentrations of MRE 3008F20 and MRE 3005F20 for 30 minutes with or without Cl-IB-MECA (10 and 100 nM) treatment. Both MRE 3008F20 and MRE 3005F20 (10 and 100 nM) were able to abrogate the effect of Cl-IB-MECA in HIF-1α modulation. As expected, neither MRE 3008F20 nor MRE 3005F20 had any effect on HIF-1β expression. FIG. 6A-B shows the results obtained with MRE 3008F20. Similar results were obtained with the antagonist MRE 3005F20 (data not shown).

We next investigated the effect of increasing concentrations of MRE 3008F20 on HIF-1α protein increase induced by a submaximal dose of Cl-IB-MECA. HIF-1α protein increase, induced by 10 nM Cl-IB-MECA, was inhibited by increasing concentrations of MRE 3008F20 (0.3-30 nM) with an IC₅₀ of 0.90±0.08 nM (FIG. 6C-D).

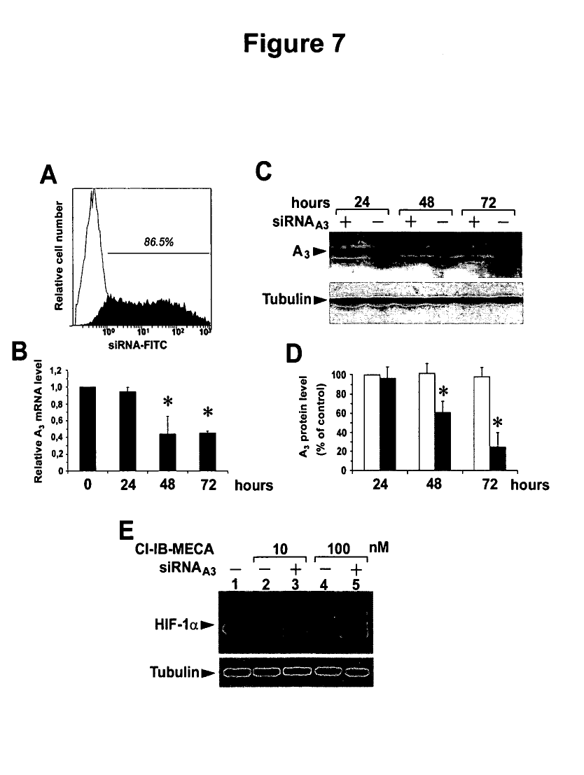

Finally, to further demonstrate that A₃ receptor is required for HIF-1α protein accumulation in response to adenosine, A375 cells were mock transfected or transfected with small interfering RNAs that target A₃ receptor mRNA (siRNA_{A3}) for degradation. To evaluate transfection efficiency A375 cells were also transfected with a siRNA control labeled with fluorescein. By flow cytometry we oberved a transfection efficiency of 85±5% (FIG. 7A). After transfection, the cells were cultured in complete media and at 24, 48 and 72 hours total RNA was isolated for Real-Time RT-PCR analysis of A₃ receptor mRNA and for Western blot analysis of A₃ receptor protein. As expected, A₃ receptor mRNA levels were significantly reduced in cells transfected with siRNA_{A3} (FIG. 7B). Furthermore, A₃ receptor protein expression was strongly reduced in siRNA_{A3}-treated cells (FIG. 7C-D). Neither mock transfection nor transfection with an siRNA targeted to an irrelevant mRNA inhibited A₃ receptor mRNA or protein expression. Therefore, at 72 hours from the siRNA_{A3} transfection, A375 cells were exposed to increasing concentrations of the A₃ adenosine receptor agonist Cl-IB-MECA (1-100 nM) for 4 hours in hypoxia. Total protein were then harvested for Western blot analysis. As control, A375 cells were exposed to RNAiFect™ Transfection reagent without siRNA_{A3}. We found that the inhibition of A₃ receptor expression is sufficient to block Cl-IB-MECA-induced HIF-1α accumulation (FIG. 7E).

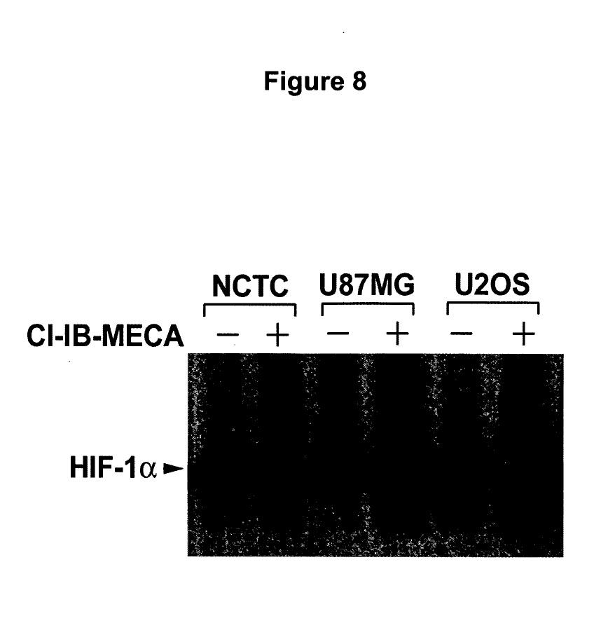

To determine whether the effect of A₃ receptor stimulation on HIF-1α expression was a general phenomenon, we assessed the ability of Cl-IB-MECA to induce HIF-1α levels in a variety of cell lines expressing A₃ adenosine receptors. After 4 hours of hypoxia under Cl-IB-MECA treatment, we were able to detect a significant increase in HIF-1α protein expression, both in human keratinocytes NCTC 2544 and in different human tumor U87MG glioblastoma and U2OS osteosarcoma cells (FIG. 8).

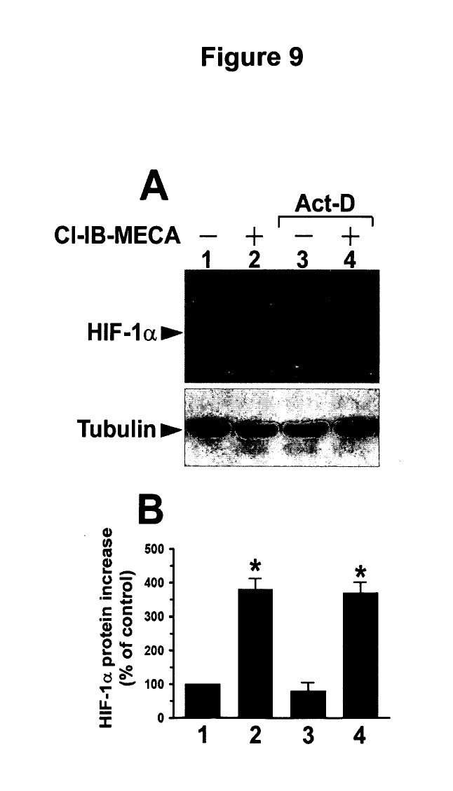

A₃ receptor mediates HIF-1α accumulation through a transcription-independent and translation-dependent pathway. To obtain a better understanding of the processes involved in HIF-1α accumulation in response to A₃ receptor stimulation in hypoxia, we investigated the effect of Cl-IB-MECA on the HIF-1α mRNA accumulation. After a treatment of A375 cells for 4 hours in hypoxia, RNA was extracted, and Real-Time RT-PCR analysis was performed. Activation of melanoma cells with 10 nM, 100 nM and 1 μM Cl-IB-MECA produced, respectively, a 1.13±0.10, 1.25±0.15 and 1.19±0.13 fold increase of HIF-1α mRNA accumulation with respect to the corresponding untreated cells, suggesting that A₃ receptor stimulation does not regulate HIF-1α mRNA transcription. To confirm this hypothesis, A375 cells were pretreated with 10 μg/ml actinomycin D (Act-D) to inhibit de novo gene transcription. Then, A375 cells were cultured for 4 hours in hypoxia in the presence of increasing concentrations of Cl-IB-MECA (100 nM). We found that A₃ receptor stimulation was able to increase HIF-1α protein expression also in the presence of Act-D (FIG. 9).

HIF-1α has been shown to be degraded through the proteasome pathway during normoxia. The enzymatic hydroxylation of proline 564 of HIF-1α controls the turnover of the protein by tagging it for interaction with the von Hippel Lindau protein (Ivan 2001; Jaakkola, 2001; Yu 2001; Maxwell 1999). When cells are hypoxic, the proline residue is not hydroxylated and HIF-1α protein accumulates. The effect of hypoxia on Pro-564 hydroxylation can be mimicked by transition metals like cobalt, iron chelators and by inhibitors of the prolyl hydroxylase enzymes (Ivan et al., 200 l; Jaakkola et al., 2001).

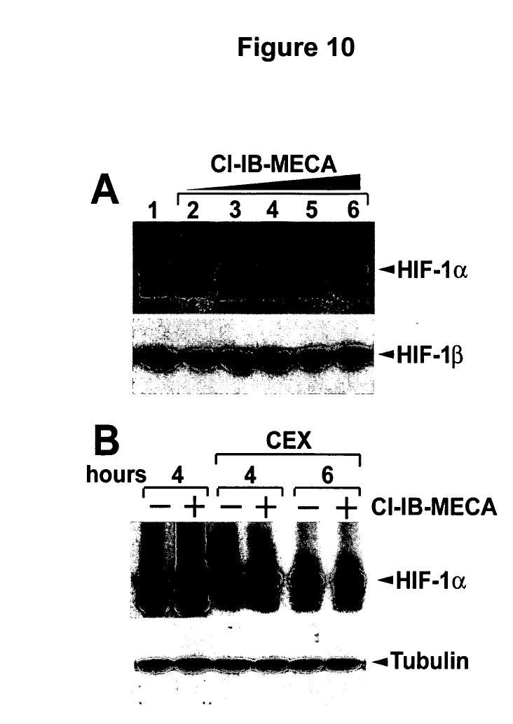

We tested the ability of A₃ adenosine receptor to modulate HIF-1α accumulation in the presence of the prolyl hydroxylase enzymes inhibitor, cobalt chloride (CoCl₂). We observed that A₃ receptor stimulation was able to increase the levels of HIF-1α protein also in CoCl₂-treated cells (FIG. 10A). To determine if A₃ receptor induces HIF-1α expression through a translation-dependent pathway, we determined HIF-1α protein modulation in the presence of the protein translation inhibitor, cycloheximide (CHX). To do this, A375 cells were cultured in normoxia for 4 hours in the presence of 100 μM CoCl₂, preventing oxygen-dependent HIF-1α protein degradation, and then A375 cells were treated with 100 nM Cl-IB-MECA in the presence or absence of CHX (1 μM). In cells exposed to CHX, Cl-IB-MECA failed to increase HIF-1α levels within 6 hours, as observed in the absence of CHX (FIG. 10B). Together, these results suggest that A₃ receptor activation increases HIF-1α protein levels through a translation-dependent pathway.

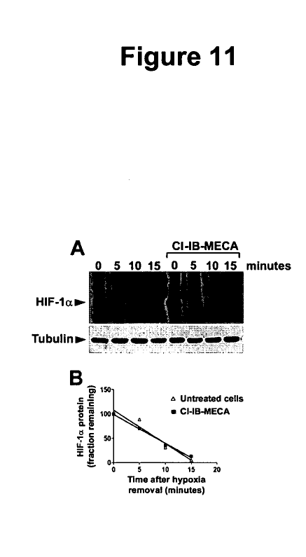

After return of hypoxic A375 cultures to normoxia the levels of HIF-1α protein decreased very rapidly and were abrogated after 15 minutes (FIG. 11A). Therefore, to study the effect of A₃ receptor activation on HIF-1α degradation, A375 cells were incubated in hypoxia in the absence and in the presence of Cl-IB-MECA 100 nM. After 4 hours, melanoma cells were exposed to normoxia and a time-course of HIF-1α disappearance was performed. Within 15 minutes after the removal of hypoxic conditions, a decrease in HIF-1α protein could be seen, in the absence and in the presence of Cl-IB-MECA with unchanged degradation rate (FIG. 1B). These results indicate that A₃ receptor activation is not able to prevent HIF-1α degradation in normoxic conditions.

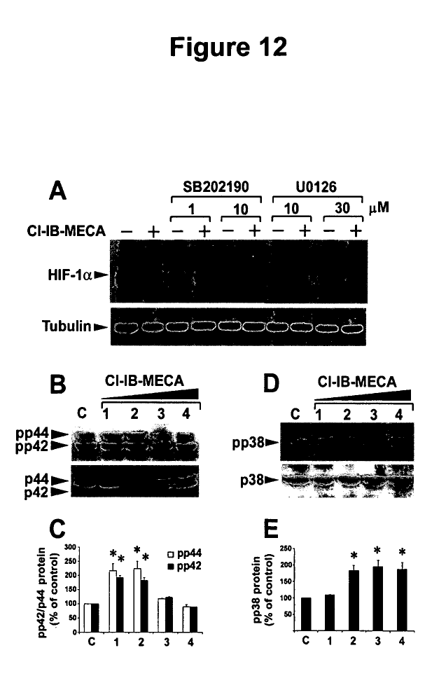

The main intracellular signaling pathways sustained by A₃ receptors during HIF-1α accumulation in hypoxia. It has been demonstrated that MAPK are involved in HIF-1α activation. To determine whether MAPK pathway was required for HIF-1α protein increase induced by A₃ receptor activation, A375 cells were pretreated with U0126, which is a potent inhibitor of MEK1/2, an upstream regulator of the phosphorylation of p44/p42 (Favata 1998), or with the inhibitor of p38 MAPK, SB202190 (Kramer 1996). Cells were then exposed to Cl-IB-MECA 100 nM for 4 hours in hypoxia, and total cellular protein extracts were prepared for immunoblot assay of HIF-1α and tubulin protein levels. As shown in FIG. 12A, both MEK inhibitor, U0126 (10 and 30 μM), and p38 MAPK inhibitor, SB202190 (1 and 10 μM), were able to inhibit Cl-IB-MECA-induced increase of HIF-1α protein expression. These results suggest that p44/p42 and p38 MAPK activity were required for the HIF-1α expression increase induced by A₃ receptor activation.

Furthermore, to confirm that p44/p42 and p38 MAPK belong to the signaling pathways fired by A₃ receptor stimulation, we also investigated endogenous p44/p42 and p38 MAPK activation levels in response to A₃ receptor agonist treatment. A375 cells were incubated with increasing concentrations of Cl-IB-MECA (1-1000 nM) for 4 hours in hypoxia, and the total cellular protein extracts were used to determine levels of phospho-p44, phospho-p42, and phospho-p38.

As shown in FIG. 12B, phosphorylation of p44 and p42 was induced in response to nanomolar concentrations of Cl-IB-MECA and the induction of p44/p42 kinases phosphorylation status was maximum by the treatment of A₃ receptor agonist 100 nM (FIG. 12C). Furthermore, we have monitored the activation levels of p38 MAPK upon A₃ receptor stimulation by the detection of its phosphorylated form on Western blot. As can be seen in FIG. 12D, a strong increase in the phosphorylation of p38 MAPK was observed after 4 hours of A₃ receptor stimulation in hypoxia. In particular, exposure of A375 cells to various concentrations of Cl-IB-MECA increased the phosphorylation of p38 MAPK in a dose-dependent manner (FIG. 12E). Phospho-p44, phospho-p42 and phospho-p38 blots were then stripped and reblotted with an antibody that equally recognizes total p44, p42 and p38 MAPKs. We found that the observed changes in phosphorylation level of p44, p42 and p38 MAPKs were not accompanied by a significative modulation in the expression levels of total proteins (FIG. 12 B-D).

DISCUSSION: To our knowledge, this is the first report which describes the role of adenosine in modulating the cellular response during hypoxia in an O₂-sensitive cell.

Hypoxia represents one of the first events in the growth of the cancer; this process creates conditions that, on one hand, are conducive to the accumulation of extracellular adenosine and, on the other hand, stabilize hypoxia-inducible factors, such as HIF-1α (Winn, 1981; Decking 1997; Ledoux 2003).

The results of the present study indicate a new way by which hypoxia may contribute to cancer development, based on the natural pathways of adenosine receptor-mediated signaling. For the first time, here we demonstrate that adenosine is able to increase HIF-1α protein expression in response to hypoxia in a dose- and time-dependent manner in A375 human melanoma cells, whereas HIF-1β protein levels are not affected.

We have previously demonstrated that all four adenosine receptors are expressed in human melanoma A375 cells (Merighi, 2001). Here, we report that A₃ receptor subtype mediates the observed adenosine effects on HIF-1α regulation in this cell line.

The effects of adenosine on HIF-1α protein accumulation are not mediated by A₁, A_{2A} or A_{2B} receptors. In support of this conclusion, DPCPX, SCH 58261 and MRE 2029F20, adenosine receptor antagonists highly selective for A₁, A_{2A} and A_{2B} receptors, respectively, did not block the stimulatory effect of adenosine on HIF-1α protein increase.

The conclusion that the effects of adenosine on HIF-1α accumulation are mediated via A₃ receptors is supported by the observation that the stimulatory effects of this nucleoside on HIF-1α protein are mimicked by the A₃ receptor agonist Cl-IB-MECA and inhibited by A₃ receptor antagonists, MRE 3008F20 and MRE 3005F20. In particular, the potencies of these drugs are in agreement with their inhibitory equilibrium binding constant (Ki) observed in binding experiments for the adenosine A₃ receptor (Merighi 2001).

Furthermore, the inhibition of A₃ receptor expression at the mRNA and protein level is sufficient to block A₃ receptor-induced HIF-1α protein accumulation. Therefore, our results indicate that the cell surface A₃ adenosine receptor transduces extracellular hypoxic signals into the cell interior. A₃ receptors are present in melanoma cells and their expression appears to be a bridge between the hypoxic insult and HIF-1α accumulation, regulating the cellular response to hypoxia, like an oxygen-sensing receptor. The extent to which A₃ receptor influences the ability of tumor cells to respond to hypoxia will require further investigation.

Similar results obtained in different cells (keratinocytes, melanoma, osteosarcoma, glioblastoma) raised the concern that the A₃ receptor stimulation effect on HIF-1α protein expression in hypoxia may be indiscriminate between normal and cancer cells, thereby demonstrating that this may be a general signaling pathway shared by many, if not all, cell types.

A₃ adenosine receptor stimulation had no effect on HIF-1α mRNA accumulation, as observed by Real-Time RT-PCR experiments. Accordingly, Act-D experiments indicate that A₃ receptor does not regulate HIF-1α protein expression through a transcription-dependent mechanism. The lack of adenosine effect on HIF-1α at transcriptional level is not surprising in view of the fact that hypoxic regulation of HIF-1α is primarily determined by stabilization of HIF-1α protein (Huang, 1998). In addition, we have obtained evidences that A₃ adenosine receptors modulate HIF-1α protein levels through a translation-dependent pathway while did not affect HIF-1α oxygen-dependent degradation. Our data suggest that A₃ adenosine receptors does not increase the half-life of HIF-1α protein while may increase the rate of HIF-1α protein synthesis, in a manner similar to the effect of various growth factors (Zhong 2000; Fukuda, 2002). Nevertheless, we cannot exclude the possibility that A₃ adenosine receptor regulates the translation of a protein, which inhibits HIF-1α degradation.

Phosphorylation and dephosphorylation activities have been suggested to be critical in the signaling pathway leading to HIF-1 activation. Several reports demonstrated that hypoxia induces the phosphorylation of HIF-1α by p44/p42 and p38 MAPKs, which increases both HIF-1α nuclear localization and transcriptional activity (Semenza 2001 ICurrOpCB; Richard 1999BBRC; Berra 2000; Richard 1999JBC; Conrad 1999; Sodhi 2000; Mottet D, 2003; Semenza 2002). In addition, adenosine has been shown to directly enhance MAPK activity in A375 human melanoma cells (Merighi et al., 2002) but also in non human cell lines stably transfected with the human A₃ receptor (Hammarberg 2004; Schulte 2000-2002-2003). In the present study, we observed that p44/p42 and p38 MAPKs are necessary to increase HIF-1α levels but also that these kinases are included in the molecular signaling pathways generated by A₃ receptor engagement. In conclusion, the present study demonstrates that adenosine, via A₃ receptors, is able to increase the levels of HIF-1α through p44/p42 and p38 MAPK pathways. Actually, further studies are needed to evaluate the role of p44/p42 and p38 MAPK in the reduced turnover, increased life and transduction of HIF-1α in hypoxia.

HIF-1α is overexpressed in tumors as a result of hypoxia and is involved in key aspects of tumor biology, such as angiogenesis, invasion and altered energy metabolism (Ratcliffe 2000). It is recognized that the inhibition of HIF-1 activity represents a novel therapeutic approach to cancer therapy, especially in combination with angiogenesis inhibitors, which would further increase intratumoral hypoxia and thus provide an even greater therapeutic window for use of an HIF-inhibitor. Recent studies indicate that pharmacologic inhibition of HIF-1α and particularly of HIF-regulated genes that are important for cancer cell survival may be more advantageous than HIF-gene inactivation therapeutic approaches (Mabjeesh et al., 2003). Many normal tissues function at pO₂ values sufficient to activate HIF, and the system has important functions under normal physiological conditions (Hopfl, 2004). This will need to be considered in the development of pharmacological inhibitors for clinical use.

Given the ability of A₃ adenosine receptor antagonists to block HIF-1α protein expression accumulation induced by adenosine, our data imply that A₃ adenosine receptor antagonists may be useful in cancer therapy. In particular, we remark that in in vivo system the extracellular fluid of solid tumors contains increased levels of adenosine (Blay et al., 1997), the endogenous agonist responsible for adenosine receptor functions. Therefore, with A₃ receptor antagonists in cancer therapy it may be possible to achieve tissue selectivity, such that a biological effect would only be observed in tumoral hypoxic cells, where high adenosine concentration increases HIF-1α accumulation.

Additional studies are warranted to determine whether A₃ receptor antagonists can block the survival of hypoxic solid tumors.

What is claimed is:

1. A method of treating an ischemic disorder in a subject in need thereof, comprising administering an effective amount of an adenosine A₃ receptor agonist, wherein the ischemic disorder is characterized by a reduction in a level of HIF-1α expression or activity.

2. The method of claim 1, wherein the level of HIF-1α expression or activity is increased by at least 10%.

3. The method of claim 1, wherein the level of HIF-1α expression or activity is increased by at least 30%.

4. The method of claim 1, wherein the level of HIF-1α expression or activity is increased by at least 60%.

5. The method of claim 1, wherein the ischemic disorder is an ischemic cardiovascular disorder, pulmonary hypertension, or a pregnancy disorder.

6. The method of claim 5, wherein the ischemic disorder is an ischemic cardiovascular disorder.

7. The method of claim 6, wherein the ischemic cardiovascular disorder is myocardial ischemia, cerebral ischemia or retinal ischemia.

8. The method of claim 5, wherein the ischemic disorder is myocardial infarction, angina, a peripheral arterial disease, deep vein thrombosis or vascular insufficiency.

9. The method of claim 5, wherein the ischemic disorder is a pregnancy disorder.

10. The method of claim 9, wherein the pregnancy disorder is preeclampsia or intrauterine growth retardation.

11. The method of claim 1, wherein the ischemic disorder is stroke or multi-infarct dementia.

12. The method of claim 1, wherein the ischemic disorder is a peripheral arterial disease.

13. The method of claim 12, wherein the peripheral arterial disease is gangrene.

14. The method of claim 1, wherein the adenosine A₃ receptor agonist is AB-MECA (N⁶-(4-amino-3-iodobenzyl)-adenosine-5′-N-methyluronamide), N⁶-2-(4-aminophenyl)ethyl-adenosine, IB-MECA) (N⁶-(3-Iodobenzyl)-5′-N-methylcarboxamidoadenosine), or 2-chloro-IB-MECA.

15. A method of preserving an organ from a mammal, comprising storing the organ in a solution comprising an effective amount of an adenosine A₃ receptor agonist.
